(19) United States
(12) Patent Application Publication
(10) Pub. No.: US 2002/0166133 A1
(43) Pub. Date: Nov. 7, 2002

(54) Neokine protein and nucleic acid molecules and uses therefor

(75) Inventors: Thomas M. Barnes, Newton, MA (US); Charles Mackay, Watertown, MA (US)

Correspondence Address: Millennium Pharmaceuticals, Inc., 75 Sidney Street, Cambridge, MA, 02139, US

(73) Assignee: Millennium Pharmaceuticals, Inc

(21) Appl. No.: 09/940,240

(22) Filed: Aug. 27, 2001

Related U.S. Application Data
(63) Continuation of application No. 09/248,239, filed on Feb. 10, 1999, now abandoned.
Application No. 09/248,239 is a continuation-in-part of application No. 09/023,664, filed on Feb. 10, 1998, now abandoned.

(51) Int. Cl. A01K 67/00; C07H 21/04; C12P 21/02; C12N 5/06; C07K 14/52

(52) U.S. Cl. 800/8; 435/69.5; 435/320.1; 435/325; 530/351; 536/23.5

(57) ABSTRACT

Novel NEOKINE polypeptides, proteins, and nucleic acid molecules are disclosed. In addition to isolated, full-length NEOKINE proteins, the invention further provides isolated NEOKINE fusion proteins, antigenic peptides and anti-NEOKINE antibodies. The invention also provides NEOKINE nucleic acid molecules, recombinant expression vectors containing a nucleic acid molecule of the invention, host cells into which the expression vectors have been introduced and non-human transgenic animals in which a NEOKINE gene has been introduced or disrupted. Diagnostic, screening and therapeutic methods utilizing compositions of the invention are also provided.

### CROSS-REFERENCE TO RELATED APPLICATIONS

The present application is a continuation-in-part of prior-filed application U.S. patent application Ser. No. 09/023,664 entitled “Novel Antikine Protein and Nucleic Acid Molecules and Uses Therefor”, filed Feb. 10, 1998. The content of the above-referenced patent application is incorporated herein by this reference in its entirety.

### BACKGROUND OF THE INVENTION

Cytokines are small peptide molecules produced by a variety of cells that mediate a wide range of biological activities. Arai et al. (1990) Annu. Rev. Biochem. 59:783 and Paul and Seder (1994) Cell 76:241. Through a complex network, cytokines regulate functions including cellular growth, inflammation, immunity, differentiation and repair. Mosmann (1991) Curr. Opin. Immunol. 3:311. One superfamily of cytokines, termed the chemokine superfamily, is a large group of more than 30 small proteins, many of which play a role in the selective recruitment and activation of leukocytes during inflammation. Wells and Peitsch (1997) J. Leukoc. Biol. 61:5. The chemokine superfamily can be subdivided into two groups based on the arrangement of the first two of four conserved cysteines, which are either separated by one amino acid (CXC chemokines) or adjacent (CC chemokines). Baggiolini et al. (1995) Int. J. Immunopharmacol. 17:2. IL-8 and the other CXC chemokines act preferentially on neutrophils, while the CC chemokines (MCP-1, MCP-2, MCP-3, RANTES, MIP-1 alpha and MIP-1 beta) act on monocytes, but not neutrophils, and have additional activities toward basophil and eosinophil granulocytes, and T-lymphocytes. Baggiolini et al., supra.

The CXC chemokine family of cytokines display disparate angiogenic activity depending upon the presence or absence of the ELR motif, a structural amino acid motif previously found to be important in receptor:ligand binding on neutrophils. CXC chemokines containing the ELR motif are potent angiogenic factors, inducing both in vitro endothelial chemotaxis and in vivo corneal neovascularization. In contrast, the CXC chemokines that lack the ELR motif including, PF4, IP-10, and MIG, not only fail to induce significant in vitro endothelial cell chemotaxis or in vivo corneal neovascularization, but are found to be potent angiostatic factors in the presence of CXC chemokines containing the ELR motif. Strieter et al. (1995) Shock 4:3. The CXC cytokines have a signature pattern which spans the region that includes the four conserved cysteine residues. A CXC-signature pattern has been generated from the consensus of multiple CXC chemokines having the following sequence C-x-C- [LIVM]-x (5,6)-[LIVMFY]-x (2)-[RKSEQ]-x-[LIVM]-x (2)-[LIVM] x(5)-[SAG]-x (2)-C-x (3)-[EQ]-[LIVM] (2)-x (9,10)-C-L-[DN], Prosite Signature PS00471.

Chemokine activities are mediated by seven-transmembrane-domain, G protein coupled receptors. To date, at least two human receptors for the CXC chemokines and at least four leukocyte receptors for the CC chemokines have been cloned. Wells et al. (1996) J. Leukoc. Biol. 59:1.

Structural analysis of chemokines has revealed that the alpha/beta structural-fold is highly conserved among both the CXC and CC chemokine classes. Although dimerization and aggregation is often observed, the chemokines function as monomers. The primary receptor-binding domain of all chemokines is near the NH2 terminus, and antagonists can be obtained by truncation or substitutions in this region. Baggiolini et al. (1997) Annu. Rev. Immunol. 15:675. A second binding site also exists in the loop that follows the two disulfides. Although well-ordered regions are not directly involved in receptor binding, together with the disulfides they provide a scaffold that determines the conformation of the sites that are critical for receptor binding. Clark-Lewis et al. (1995) J. Leukoc. Biol. 57:5.

Recently, some of the chemokine receptors and ligands have been implicated in the mechanism of viral infection for primate lentiviruses such as HIV-1 in addition to their more general role as leukocyte attractants. Wells and Peitsch, supra.

Given the important role of chemokine receptors and ligands, such as the CXC and CC chemokines, in regulating inflammation and viral infection, there exists a need for identifying novel chemokines and their receptors as well as for modulators of such molecules for use in regulating a variety of cellular responses.

### SUMMARY OF THE INVENTION

The present invention is based, at least in part, on the discovery of nucleic acid molecules which encode a novel family of secreted proteins, referred to herein as the Neokine family of proteins (“NEOKINES” or “NEOKINE proteins”) which are ligands for a previously-identified putative G protein-coupled receptor termed “RDC1” also referred to herein as the “NEOKINE receptor”. The NEOKINE molecules of the present invention are useful as modulating agents in regulating a variety of cellular processes. Accordingly, in one aspect, this invention provides isolated nucleic acid molecules encoding NEOKINE proteins or biologically active portions thereof, as well as nucleic acid fragments suitable as primers or hybridization probes for the detection of NEOKINE-encoding nucleic acids.

In one embodiment, a NEOKINE nucleic acid molecule is 60% homologous to the nucleotide sequence shown in SEQ ID NO:1, SEQ ID NO:3, the nucleotide sequence of the DNA insert of the plasmid deposited with ATCC as Accession Number 98751, or complement thereof. In another embodiment, a NEOKINE nucleic acid molecule is 60% homologous to the nucleotide sequence shown in SEQ ID NO:4, SEQ ID NO:6, or a complement thereof. In yet another embodiment, a NEOKINE nucleic acid molecule is 60% homologous to the nucleotide sequence shown in SEQ ID NO:7 or SEQ ID NO:9. In yet another embodiment, a NEOKINE nucleic acid molecule is 60% homologous to the nucleotide sequence shown in SEQ ID NO: 10. In a preferred embodiment, an isolated NEOKINE nucleic acid molecule has the nucleotide sequence shown SEQ ID NO:3, or a complement thereof. In another embodiment, a NEOKINE nucleic acid molecule further comprises nucleotides 1-96 of SEQ ID NO: 1. In another embodiment, a NEOKINE nucleic acid molecule further comprises nucleotides 394-1564 of SEQ ID NO:1. In another preferred embodiment, an isolated NEOKINE nucleic acid molecule has the nucleotide sequence shown in SEQ ID NO:1.

In another preferred embodiment, an isolated NEOKINE nucleic acid molecule has the nucleotide sequence shown SEQ ID NO:6, or a complement thereof. In another embodiment, a NEOKINE nucleic acid molecule further comprises nucleotides 1-211 of SEQ ID NO:4. In another embodiment, a NEOKINE nucleic acid molecule further comprises nucleotides 509-1656 of SEQ ID NO:4. In another preferred embodiment, an isolated NEOKINE nucleic acid molecule has the nucleotide sequence shown in SEQ ID NO:4.

In another preferred embodiment, an isolated NEOKINE nucleic acid molecule has the nucleotide sequence shown SEQ ID NO:9, or a complement thereof. In another embodiment, a NEOKINE nucleic acid molecule further comprises nucleotides 235-1372 of SEQ ID NO:7. In another preferred embodiment, an isolated NEOKINE nucleic acid molecule has the nucleotide sequence shown in SEQ ID NO:7.

In another preferred embodiment, an isolated NEOKINE nucleic acid molecule has the nucleotide sequence shown SEQ ID NO:22, or a complement thereof. In another embodiment, a NEOKINE nucleic acid molecule further comprises nucleotides 285-1458 of SEQ ID NO:10. In another preferred embodiment, an isolated NEOKINE nucleic acid molecule has the nucleotide sequence shown in SEQ ID NO: 10.

In another preferred embodiment, an isolated NEOKINE nucleic acid molecule is of human origin and has the nucleotide sequence of the DNA insert of the plasmid deposited with ATCC as Accession Number 98751, or a complement thereof. In another embodiment, an isolated NEOKINE nucleic acid molecule is of rat origin. In another embodiment, an isolated NEOKINE nucleic acid molecule is of macaque origin.

In another embodiment, a NEOKINE nucleic acid molecule includes a nucleotide sequence encoding a protein having an amino acid sequence sufficiently homologous to the amino acid sequence of SEQ ID NO:2, SEQ ID NO:5, SEQ ID NO:8, or SEQ ID NO:11. In another preferred embodiment, a NEOKINE nucleic acid molecule includes a nucleotide sequence encoding a protein having an amino acid sequence at least 60% homologous to the amino acid sequence of SEQ ID NO:2. In yet another preferred embodiment, a NEOKINE nucleic acid molecule includes a nucleotide sequence encoding a protein having an amino acid sequence at least 60% homologous to the amino acid sequence of SEQ ID NO:5. In yet another preferred embodiment, a NEOKINE nucleic acid molecule includes a nucleotide sequence encoding a protein having an amino acid sequence at least 60% homologous to the amino acid sequence of SEQ ID NO:8. In yet another preferred embodiment, a NEOKINE nucleic acid molecule includes a nucleotide sequence encoding a protein having an amino acid sequence at least 60% homologous to the amino acid sequence of SEQ ID NO:11.

In another embodiment, an isolated nucleic acid molecule of the present invention encodes a NEOKINE protein which includes a NEOKINE CXC signature motif. In another embodiment, an isolated nucleic acid molecule of the present invention encodes a NEOKINE protein which includes a NEOKINE CXC signature motif and a signal sequence and is secreted. In yet another embodiment, a NEOKINE nucleic acid molecule encodes a NEOKINE protein and is a naturally occurring nucleotide sequence.

Another embodiment of the invention features NEOKINE nucleic acid molecules which specifically detect NEOKINE nucleic acid molecules relative to nucleic acid molecules encoding non-NEOKINE proteins. For example, in one embodiment, a NEOKINE nucleic acid molecule is at least 650 nucleotides in length and hybridizes under stringent conditions to a nucleic acid molecule comprising the nucleotide sequence shown in SEQ ID NO:1, SEQ ID NO:4, SEQ ID NO:7, or SEQ ID NO:10, or a complement thereof.

Another embodiment of the invention provides an isolated nucleic acid molecule which is antisense to the coding strand of a NEOKINE nucleic acid.

Another aspect of the invention provides a vector comprising a NEOKINE nucleic acid molecule. In certain embodiments, the vector is a recombinant expression vector. In another embodiment, the invention provides a host cell containing a vector of the invention. The invention also provides a method for producing a NEOKINE protein by culturing in a suitable medium, a host cell of the invention containing a recombinant expression vector such that a NEOKINE protein is produced.

Another aspect of this invention features isolated or recombinant NEOKINE proteins and polypeptides. In one embodiment, an isolated NEOKINE protein includes a NEOKINE CXC signature motif and is secreted. In another embodiment, an isolated NEOKINE protein includes a NEOKINE CXC signature motif and a signal sequence, and is secreted. In another embodiment, an isolated NEOKINE protein has an amino acid sequence sufficiently homologous to the amino acid sequence of SEQ ID NO:2, SEQ ID NO:5, SEQ ID NO:8, or SEQ ID NO:11. In a preferred embodiment, a NEOKINE protein has an amino acid sequence at least about 60% homologous to the amino acid sequence of SEQ ID NO:2. In another preferred embodiment, a NEOKINE protein has an amino acid sequence at least about 60% homologous to the amino acid sequence of SEQ ID NO:5. In another preferred embodiment, a NEOKINE protein has an amino acid sequence at least about 60% homologous to the amino acid sequence of SEQ ID NO:8. In another preferred embodiment, a NEOKINE protein has an amino acid sequence at least about 60% homologous to the amino acid sequence of SEQ ID NO:11. In another embodiment, a NEOKINE protein has the amino acid sequence of SEQ ID NO:2, SEQ ID NO:5, SEQ ID NO:8, or SEQ ID NO:11.

Another embodiment of the invention features an isolated NEOKINE protein which is encoded by a nucleic acid molecule having a nucleotide sequence at least about 60% homologous to a nucleotide sequence of SEQ ID NO:1, or a complement thereof. Another embodiment of the invention features an isolated NEOKINE protein which is encoded by a nucleic acid molecule having a nucleotide sequence at least about 60% homologous to a nucleotide sequence of SEQ ID NO:4, or a complement thereof. Another embodiment of the invention features an isolated NEOKINE protein which is encoded by a nucleic acid molecule having a nucleotide sequence at least about 60% homologous to a nucleotide sequence of SEQ ID NO:7, or a complement thereof. Another embodiment of the invention features an isolated NEOKINE protein which is encoded by a nucleic acid molecule having a nucleotide sequence at least about 60% homologous to a nucleotide sequence of SEQ ID NO:10, or a complement thereof. This invention further features an isolated NEOKINE protein which is encoded by a nucleic acid molecule having a nucleotide sequence which hybridizes under stringent hybridization conditions to a nucleic acid molecule comprising the nucleotide sequence of SEQ ID NO:1, SEQ ID NO:4, SEQ ID NO:7, SEQ ID NO:10, or a complement thereof.

The NEOKINE proteins of the present invention, or biologically active portions thereof, can be operatively linked to a non-NEOKINE polypeptide to form NEOKINE fusion proteins. The invention further features antibodies that specifically bind NEOKINE proteins, such as monoclonal or polyclonal antibodies. In addition, the NEOKINE proteins or biologically active portions thereof can be incorporated into pharmaceutical compositions, which optionally include pharmaceutically acceptable carriers.

In another aspect, the present invention provides a method for detecting NEOKINE expression in a biological sample by contacting the biological sample with an agent capable of detecting a NEOKINE nucleic acid molecule, protein or polypeptide such that the presence of a NEOKINE nucleic acid molecule, protein or polypeptide is detected in the biological sample.

In another aspect, the present invention provides a method for detecting the presence of NEOKINE activity in a biological sample by contacting the biological sample with an agent capable of detecting an indicator of NEOKINE activity such that the presence of NEOKINE activity is detected in the biological sample.

In another aspect, the invention provides a method for modulating NEOKINE activity comprising contacting the cell with an agent that modulates NEOKINE activity such that NEOKINE activity in the cell is modulated. In one embodiment, the agent inhibits NEOKINE activity. In another embodiment, the agent stimulates NEOKINE activity. In one embodiment, the agent is an antibody that specifically binds to a NEOKINE protein. In another embodiment, the agent modulates expression of NEOKINE by modulating transcription of a NEOKINE gene or translation of a NEOKINE mRNA. In yet another embodiment, the agent is a nucleic acid molecule having a nucleotide sequence that is antisense to the coding strand of a NEOKINE mRNA or a NEOKINE gene.

In one embodiment, the methods of the present invention are used to treat a subject having a disorder characterized by aberrant NEOKINE protein or nucleic acid expression or activity by administering an agent which is a NEOKINE modulator to the subject. In one embodiment, the NEOKINE modulator is a NEOKINE protein. In another embodiment the NEOKINE modulator is a NEOKINE nucleic acid molecule. In yet another embodiment, the NEOKINE modulator is a peptide, peptidomimetic, or other small molecule. In a preferred embodiment, the disorder characterized by aberrant NEOKINE protein or nucleic acid expression is a developmental, differentiative, proliferative disorder, an immunological disorder, or cell death.

The present invention also provides a diagnostic assay for identifying the presence or absence of a genetic alteration characterized by at least one of (i) aberrant modification or mutation of a gene encoding a NEOKINE protein; (ii) mis-regulation of said gene; and (iii) aberrant post-translational modification of a NEOKINE protein, wherein a wild-type form of said gene encodes an protein with a NEOKINE activity.

The present invention also provides methods for identifying compounds which modulate binding of NEOKINE to the NEOKINE receptor and methods for identifying compounds which modulate the activity of the NEOKINE receptor (e.g., cell-based as well as in vitro screening assays).

Other features and advantages of the invention will be apparent from the following detailed description and claims.

### BRIEF DESCRIPTION OF THE DRAWINGS

FIG. 1 depicts the cDNA sequence and predicted amino acid sequence of human NEOKINE-1. The nucleotide sequence corresponds to nucleic acids 1 to 1564 of SEQ ID NO:1. The amino acid sequence corresponds to amino acids 1 to 99 of SEQ ID NO:2.

FIG. 2 depicts the cDNA sequence and predicted amino acid sequence of murine NEOKINE-1. The nucleotide sequence corresponds to nucleic acids 1 to 1656 of SEQ ID NO:4. The amino acid sequence corresponds to amino acids 1 to 99 of SEQ ID NO:5.

FIG. 3 depicts the partial cDNA sequence and partial predicted amino acid sequence of rat NEOKINE-1. The nucleotide sequence corresponds to nucleic acids 1 to 1372 of SEQ ID NO:7. The amino acid sequence corresponds to amino acids 1 to 79 of SEQ ID NO:8.

FIG. 4 depicts the partial cDNA sequence of macaque NEOKINE-1. The nucleotide sequence corresponds to nucleic acids 1 to 1458 of SEQ ID NO:10. The amino acid sequence corresponds to amino acids 1 to 94 of SEQ ID NO:21.

FIG. 5 is a diagram depicting the the relationship between the NEOKINE proteins of the instant invention. The figure depicts the functional domains of the NEOKINE family members, human NEOKINE-1 (SEQ ID NO:2), mouse NEOKINE-1 (SEQ ID NO:5), rat NEOKINE-1 (SEQ ID NO:8), and macaque NEOKINE-1 (SEQ ID NO:21). The NEOKINE CXC signature motifs are indicated in italics. The conserved cysteine residues are indicated with asterisks.

FIG. 6 depicts a structure similarity diagram of chemokines and their receptors.

FIG. 7 depicts an alignment of the nucleotide sequences of human, murine and canine RDC1/GPRN1.

FIG. 8 depicts an alignment of the amino acid sequences of human, murine and canine RDC1/GPRN1.

### DETAILED DESCRIPTION OF THE INVENTION

The present invention is based on the discovery of family of molecules, referred to herein as NEOKINE protein and nucleic acid molecules. The NEOKINEs are members of the non-ELR-CXC subfamily of chemokines (a shortening of chemoattractant cytokines). The CXC-chemokines display four highly conserved cysteine amino acid residues, with the first two cysteines separated by one non-conserved amino acid residue. The cloning of the NEOKINE family of CXC chemokines revealed at least three atypical features which distinguish them from previously characterized chemokines. These are (1) the presence of approximately 5 residues between the third and fourth conserved cysteine residues which are absent from other CXC chemokines; (2) the fewest residues preceeding the predicted amino terminus of the mature form of any naturally-occurring chemokine; and (3) a general dissimilarity to all other chemokines in the region between the second and third conserved cysteines. Phylogenetic analysis of known chemokines (e.g., known CXC as well as CC chemokines) further demonstrates that NEOKINE is unique from other chemokines subfamilies identified to date although it is clearly a related chemokine.

The family of NEOKINE chemokines described herein include human, murine, rat and macaque NEOKINE-1. Upon comparison of the sequences for each species orthologue, it was noticed that the NEOKINE-1 chemokines displayed a remarkable degree of identity between orthologues. For example, human NEOKINE-1 is 92% identical at the amino acid level to murine NEOKINE-1 (as determined using a Lipman-Pearson algorithm (Lipman and Pearson (1985) Science 227:1435-1441, Ktuple=2, Gap Penalty=4, Gap Length Penalty=12).

The present invention is also based on the discovery that NEOKINE is the surrogate ligand for a previously-identified orphan receptor known in the art as RDC1. RDC1 was first identified as one of four orphan receptors cloned from a dog thyroid cDNA library based on homology to the seven-transmembrane helice-containing G-protein coupled receptors (Libert et al. (1989) Science 244:569-572). Three of these, RDC4, RDC7, and RDC8 have since been identified as 5-HT_{1D}, adenosine A₁ and adenosine A₂ receptors, respectively (Maenhaut et al. (1991) Biochem. Biophys. Res. Commun. 173:1169-1178; Libert et al. (1991) EMBO J. 10:1677-1682; and Maenhaut et al. (1991) Biochem. Biophys. Res. Commun. 180:1460-1468). A human orthologue of the fourth orphan, termed GPRN1, was subsequently cloned which was initially proposed to be the receptor for vasoactive intestinal peptide (“VIP”) (Sreedharan et al. (1991) Proc. Natl. Acad. Sci. USA 88:4986-4990). This finding has more recently been refuted (Nagata et al. (1992) Trends Biochem. Sci. 13:102-103; and Cook et al. (1992) FEBS Lett. 300:149-152), leaving the receptor orphaned. Recent reports have characterized the tissue expression of RDC1/GPRN1 receptor (Law and Rosenzweig (1994) Biochem. Biophys. Res. Commun. 201:458-465) and have identified and characterized a murine homologue of RDC1/GPRN1 (Heesen et al. (1998) Immunogenetics 47:364-370).

Sequence comparison of human and murine RDC1/GPRN1 reveals that these orthologues exhibit remarkable sequence identity as is the case with species orthologues of NEOKINE. Moreover, phylogenetic analysis of chemokine receptors demonstrates that RDC1/GPRN1 is a unique but distantly related member of the CXC subfamily of chemokine receptors. Given the remarkable sequence identity between species orthologues of NEOKINE and between orthologues of RDC1/GPRN1, as well as the unique positions which both NEOKINE and RDC1/GPRN1 fall on their respective phylogenetic trees, it has been determined that the NEOKINE is the surrogate ligand for the previously orphaned RDC1/GPRN1 receptor.

For convenience, certain terms employed in the specification, examples and claims are defined below.

The term “family” when referring to the protein and nucleic acid molecules of the invention is intended to mean two or more proteins or nucleic acid molecules having a common structural domain and having sufficient amino acid or nucleotide sequence homology as defined herein. Such family members can be naturally occurring and can be from either the same or different species. For example, a family can contain a first protein of human origin, as well as other, distinct proteins of human origin or alternatively, can contain homologues of non-human origin. Members of a family may also have common functional characteristics.

In one embodiment, the NEOKINE proteins of the present invention are proteins having an amino acid sequence of about 75-125, preferably about 80-120, more preferably about 85-115, more preferably about 90-110, and even more preferably about 95-105 amino acids containing 3-7, preferably 5-6, and more preferably 4 cysteine residues which are conserved between family members. In another embodiment, a NEOKINE family member is identified based on the presence of at least one “NEOKINE CXC signature motif” in the protein or corresponding nucleic acid molecule. As used herein, the term “NEOKINE CXC signature motif” refers to a protein domain having an amino acid sequence of about 35-65, preferably about 40-60, more preferably about 45-55 amino acid residues, and even more preferably at least about 48-50 amino acids containing 3-7, preferably 5-6, and more preferably 4 cysteine residues which are conserved between family members, the first three residues of the NEOKINE CXC signature motif having the sequence C-X-C (“CXC”), wherein X is any amino acid and C is cysteine. In a preferred embodiment, a NEOKINE CXC signature motif has the pattern X (0-2)-C-X-C-X (20-24)-C-X (17-24)-C-X (0-2), wherein X is any amino acid and C is cysteine. In another preferred embodiment, a NEOKINE CXC signature motif has the pattern X (0-2)-C-X-C-X (23, 24)-C-X (20, 21)-C-X (2), wherein X is any amino acid and C is cysteine. In another preferred embodiment, a NEOKINE CXC signature motif has the pattern X (0-2)-C-X-C-X (6,7)-[LIVMFY]-X (2)-[RKSEQ]-X-[LIVM]-X (2)-[LIVM]-X (8)-C-X (4)-[LIVM] (2)-X (13,14)-C-[LIVM]-X. In another preferred embodiment, a NEOKINE CXC signature motif has the pattern X (0, 1)-[RK]-C-[RK]-C-X(4)-P-X(4, 5)-[ED]-X(6)-[KR]-X(5)-C-[DE](2)-X-[LIVMFY](4)-X (12,13)-H-C-[LIVM]-H. The motifs described herein, are described according to standard Prosite Signature designation (e.g., X (0-2) designates any amino acid, n=0-2; X (6, 7) designates any amino acid, n=6 or 7; and [LIVM] indicates any one of Leu, Ile, Val, or Met. All amino acids are described using universal single letter abbreviations according to these motifs. In a preferred embodiment, the N-terminal amino acid of the NEOKINE CXC signature motif is the N-terminal amino acid of the mature NEOKINE protein.

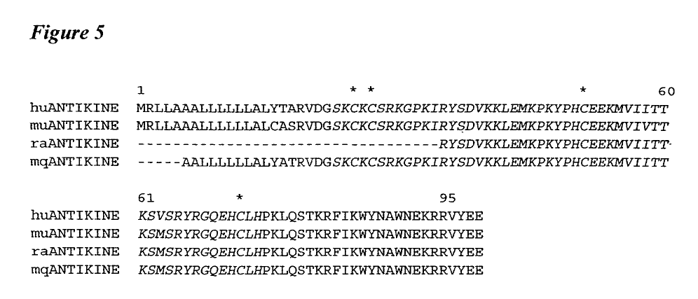

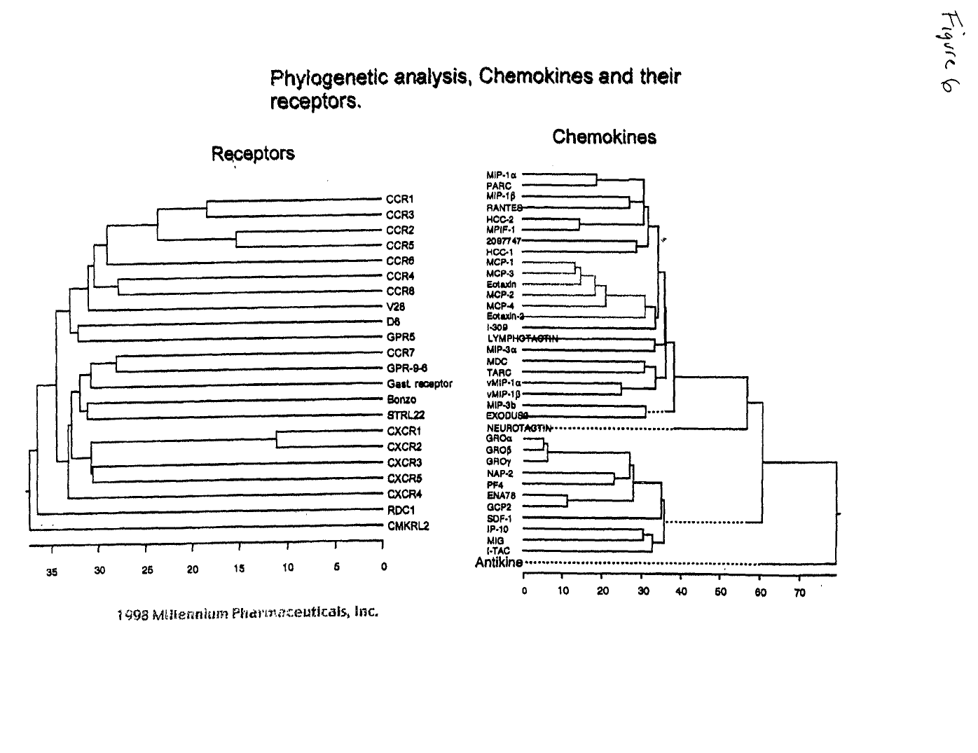

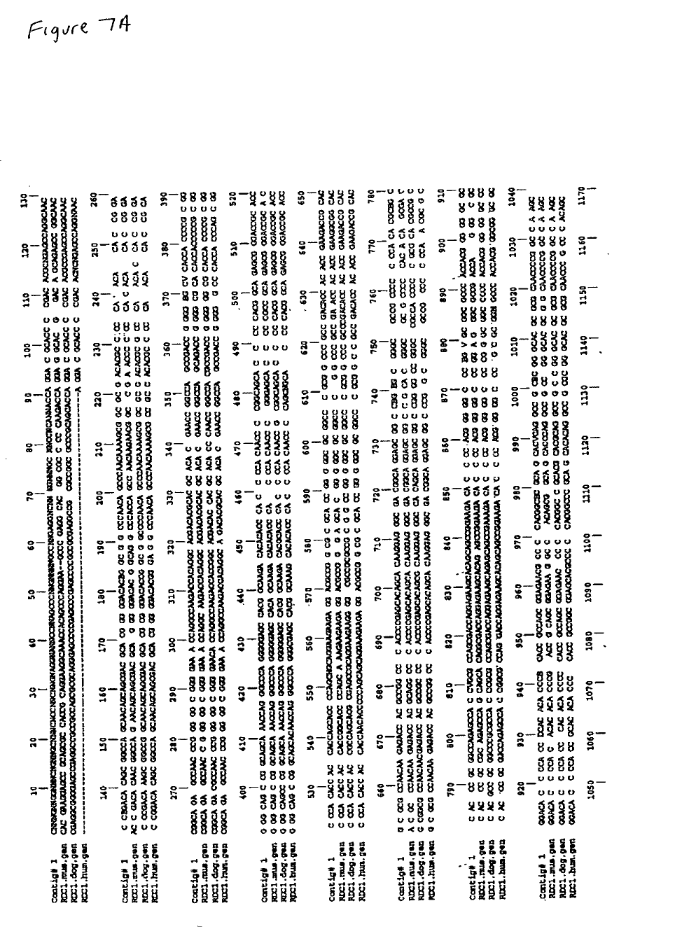

Accordingly, in one embodiment, a NEOKINE protein is human NEOKINE-1 which contains a NEOKINE CXC signature motif containing about amino acids 25-72 of SEQ ID NO:2 having the sequence CXC at amino acid residues 25-27, and having 4 conserved cysteine residues at the positions indicated in FIG. 5. In another embodiment, a NEOKINE protein is murine NEOKINE-1 which contains a NEOKINE CXC signature motif containing about amino acids 25-72 of SEQ ID NO:5 having the sequence CXC at amino acid residues 25-27, and having 4 conserved cysteine residues at the positions indicated in FIG. 5. In another embodiment, a NEOKINE protein is rat NEOKINE-1 which contains a NEOKINE CXC signature motif containing at least amino acids 4-51 of SEQ ID NO:8, having 4 conserved cysteine residues at the positions indicated in FIG. 5. In another embodiment, a NEOKINE protein is macaque NEOKINE-1 which contains a NEOKINE CXC signature motifcontaining at least amino acids 20-67 of SEQ ID NO:21, having 4 conserved cysteine residues at the positions indicated in FIG. 5.

In another embodiment of the invention, a NEOKINE protein has at least one NEOKINE CXC signature motif and a signal sequence. As used herein, a “signal sequence” refers to a peptide containing about 20 amino acids which occurs at the N-terminus of secretory and integral membrane proteins and which contains a majority of hydrophobic amino acid residues. For example, a signal sequence contains at least about 14-28 amino acid residues, preferably about 16-26 amino acid residues, more preferably about 18-24 amino acid residues, and more preferably about 20-22 amino acid residues, and has at least about 40-70%, preferably about 50-65%, and more preferably about 55-60% hydrophobic amino acid residues (e.g., Alanine, Valine, Leucine, Isoleucine, Phenylalanine, Tyrosine, Tryptophan, or Proline). Such a “signal sequence”, also referred to in the art as a “signal peptide”, serves to direct a protein containing such a sequence to a lipid bilayer. For example, in one embodiment, a NEOKINE-1 protein contains a signal sequence of about amino acids 1-22 of SEQ ID NO:2. In another embodiment, a NEOKINE-2 protein contains a signal sequence of about amino acids 1-22 of SEQ ID NO:5. In another embodiment, a NEOKINE-2 protein contains a signal sequence of about amino acids 1-17 of SEQ ID NO:21.

Accordingly, one embodiment of the invention features a NEOKINE protein having at least a NEOKINE CXC signature motif. Another embodiment features a NEOKINE protein having at least a NEOKINE CXC signature motif and a signal peptide.

Preferred NEOKINE molecules of the present invention have an amino acid sequence sufficiently homologous to the amino acid sequence of SEQ ID NO:2, SEQ ID NO:5, SEQ ID NO:8, or SEQ ID NO:11. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains and/or a common functional activity. For example, amino acid or nucleotide sequences which share common structural domains have at least about 50% homology, preferably 60% homology, more preferably 70%-80%, and even more preferably 90-95% homology across the amino acid sequences of the domains and contain at least one and preferably two structural domains, are defined herein as sufficiently homologous. Furthermore, amino acid or nucleotide sequences which share at least 50%, preferably 60%, more preferably 70-80, or 90-95% homology and share a common functional activity are defined herein as sufficiently homologous.

As used interchangeably herein, an “NEOKINE activity”, “biological activity of NEOKINE” or “functional activity of NEOKINE”, refers to an activity exerted by a NEOKINE protein, polypeptide or nucleic acid molecule on a NEOKINE responsive cell as determined in vivo, or in vitro, according to standard techniques. In one embodiment, a NEOKINE activity is a direct activity, such as an association with a NEOKINE-target molecule. As used herein, a “target molecule” or “binding partner” is a molecule with which a NEOKINE protein binds or interacts in nature, such that NEOKINE-mediated function is acheived. A NEOKINE target molecule can be a non-NEOKINE molecule or a NEOKINE protein or polypeptide of the present invention. In an exemplary embodiment, a NEOKINE target molecule is a carbohydrate molecule on the cell membrane (e.g., heparan sulfate). In another exemplary embodiment, a NEOKINE target molecule is a membrane-bound protein (e.g., a “NEOKINE receptor”).

In another embodiment, a NEOKINE target is a membrane-bound chemokine receptor. In another embodiment, a NEOKINE target is an protein molecule (e.g., a “NEOKINE binding partner”). In such an exemplary embodiment, a NEOKINE binding partner can be an non-NEOKINE protein or a second NEOKINE protein molecule of the present invention. Alternatively, a NEOKINE activity is an indirect activity, such as a cellular signaling activity mediated by interaction of the NEOKINE protein with a second protein (e.g., a NEOKINE receptor or a receptor specific for another chemokine).

In one embodiment, a NEOKINE activity is at least one or more of the following activities: (i) interaction of a NEOKINE protein with a membrane-bound NEOKINE receptor (e.g., RDC1); (ii) interaction of a NEOKINE protein with a membrane-bound chemokine receptor; (iii) indirect interaction of a NEOKINE protein with an intracellular protein via a membrane-bound NEOKINE receptor (e.g., RDC1); (iv) indirect interaction of a NEOKINE protein with an intracellular protein via a membrane-bound chemokine receptor; (v) complex formation between a soluble NEOKINE protein and a NEOKINE binding partner; (vi) inhibition of the interaction of chemokines (e.g., pro-inflammatory chemokines) by binding to their cognate receptors; (vii) inhibition of the binding of HIV to HIV co-receptors; and (vii) inhibition of the binding of HIV to HIV co-receptors wherein said binding induces subsequent infection of susceptible cells.

In another embodiment, a NEOKINE activity is at least one or more of the following activities: (1) modulation of cellular signal transduction, either in vitro or in vivo; (2) regulation of gene transcription in a cell expressing a NEOKINE receptor or a chemokine receptor; (3) regulation of gene transcription in a cell expressing a NEOKINE receptor (e.g., RDC1) or a chemokine receptor, wherein said cell is involved in angiogenesis or inflanmmation; (4) regulation of angiogenesis; (5) regulation of angiogenesis, wherein said regulation comprises inhibibition of angiogenesis; (6) regulation of angiogenesis, wherein said regulation comprises maintenance of angiostasis; (7) regulation of inflammation; (8) inhibition of chemoattraction (e.g., neutrophil chemoattraction); and (9) inhibition of pro-inflammatory chemokines by binding to their cognate receptors.

In a preferred embodiment of the invention a NEOKINE or NEOKINE modulator is useful for regulating, preventing and/or treating at least one or more of the following proliferative diseases or disorders: (1) cancers of the epithelia (e.g., carcinomas of the pancreas, stomach, liver, secretory glands (e.g., adenocarcinoma) bladder, lung, breast, skin (e.g., malignant melanoma), reproductive tract including prostate gland, ovary, cervix and uterus); (2) cancers of the hematopoietic and immune system (e.g., leukemias and lymphomas); (3) cancers of the central nervous, brain system and eye (e.g., gliomas, neuroblastoma and retinoblastoma); and (4) cancers of connective tissues, bone, muscles and vasculature (e.g., sarcomas).

In yet another embodiment of the invention, a NEOKINE or NEOKINE modulator is useful for regulating, preventing and/or treating at least one or more of the following diseases or disorders: (1) inflammation; (2) psoriasis; (3) immune rejection following skin graft; (4) immune rejection following kidney transplant; (5) kidney inflammation in acute renal failure; (6) brain inflammation following stroke or ischaemia; and (7) brain inflammation following viral infection.

Accordingly, another embodiment of the invention features isolated NEOKINE proteins and polypeptides having a NEOKINE activity. Preferred NEOKINE proteins have at least one NEOKINE CXC signature motif and a NEOKINE activity. In another preferred embodiment, a NEOKINE protein further comprises a signal sequence. In still another preferred embodiment, a NEOKINE protein has a NEOKINE CXC signature motif, a NEOKINE activity, and an amino acid sequence sufficiently homologous to an amino acid sequence of SEQ ID NO:2, SEQ ID NO:5, SEQ ID NO:8, or SEQ ID NO:11.

The human NEOKINE-1 cDNA, which is approximately 1564 nucleotides in length, encodes a protein which is approximately 99 amino acid residues in length. The human NEOKINE-1 protein contains at least a NEOKINE CXC signature motif. A NEOKINE CXC signature motif can be found at least, for example, from about amino acids 25-72 of SEQ ID NO:2. The human NEOKINE-1 protein is predicted to be a secreted protein which further contains a signal sequence at about amino acids 1-22 of SEQ ID NO:2. The prediction of such a signal peptide can be made, for example, utilizing the computer algorithm SIGNALP (Henrik, et al. (1997) Protein Engineering 10:1-6).

The murine NEOKINE-1 cDNA, which is approximately 1564 nucleotides in length, encodes approximately 99 amino acid residues of the murine NEOKINE-1 protein. The murine NEOKINE-1 protein contains a NEOKINE CXC signature motif. A NEOKINE CXC signature motif can be found at least, for example, from about amino acids 25-72 of SEQ ID NO:5. The murine NEOKINE-1 protein is predicted to be a secreted protein which further contains a signal sequence at about amino acids 1-22 of SEQ ID NO:5.

The rat NEOKINE-1 cDNA, which is approximately 1372 nucleotides in length, encodes approximately 79 amino acid residues of the rat NEOKINE-1 protein. The rat NEOKINE-1 protein contains a NEOKINE CXC signature motif. A NEOKINE CXC signature motif comprises at least about amino acids 4-51 of SEQ ID NO:8. The rat NEOKINE-1 protein is predicted to be a secreted protein.

The macaque NEOKINE-1 cDNA, which is approximately 1458 nucleotides, encodes approximately 94 amino acid residues of the macaque NEOKINE-1 protein. The macaque NEOKINE-1 protein contains a NEOKINE CXC signature motif. A NEOKINE CXC signature motif comprises at least about amino acids 20-67 of SEQ ID NO:21. The macaque NEOKINE-1 protein is predicted to be a secreted protein which further contains a signal sequence including at least amino acids 1-17 of SEQ ID NO:21.

Various aspects of the invention are described in further detail in the following subsections:

I. Isolated Nucleic Acid Molecules

One aspect of the invention pertains to isolated nucleic acid molecules that encode NEOKINE proteins or biologically active portions thereof, as well as nucleic acid fragments sufficient for use as hybridization probes to identify NEOKINE-encoding nucleic acids (e.g., NEOKINE mRNA) and fragments for use as PCR primers for the amplification or mutation of NEOKINE nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated NEOKINE nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. An isolated chromosome is not an isolated nucleic acid molecule as defined herein. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, SEQ ID NO:4, SEQ ID NO:7, SEQ ID NO:10, or the nucleotide sequence of the DNA insert of the plasmid deposited with ATCC as Accession Number 98751, or a portion thereof, can be isolated using standard molecular biology techniques and the sequence information provided herein. Using all or portion of the nucleic acid sequence of SEQ ID NO:1, SEQ ID NO:4, SEQ ID NO:7, SEQ ID NO:10, or the nucleotide sequence of the DNA insert of the plasmid deposited with ATCC as Accession Number 98751, NEOKINE nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, SEQ ID NO:4, SEQ ID NO:7, SEQ ID NO:10, or the nucleotide sequence of the DNA insert of the plasmid deposited with ATCC as Accession Number 98751, can be isolated by the polymerase chain reaction (PCR) using synthetic oligonucleotide primers designed based upon the sequence of SEQ ID NO:1, SEQ ID NO:4, SEQ ID NO:7, SEQ ID NO:10, or the nucleotide sequence of the DNA insert of the plasmid deposited with ATCC as Accession Number 98751.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NEOKINE nucleotide sequences can be prepared by standard synthetic techniques, e.g., using an automated DNA synthesizer.

In a preferred embodiment, an isolated nucleic acid molecule of the invention comprises the nucleotide sequence shown in SEQ ID NO:1. The sequence of SEQ ID NO:1 corresponds to the human NEOKINE-1 cDNA. This cDNA comprises sequences encoding the human NEOKINE-1 protein (i.e., “the coding region”, from nucleotides 97-393), as well as 5+ untranslated sequences (nucleotides 1-97) and 3′ untranslated sequences (nucleotides 394-1564). Alternatively, the nucleic acid molecule can comprise only the coding region of SEQ ID NO:1 (e.g., nucleotides 97-393, corresponding to SEQ ID NO:3).

In another preferred embodiment, an isolated nucleic acid molecule of the invention comprises the nucleotide sequence shown in SEQ ID NO:4. The sequence of SEQ ID NO:4 corresponds to a murine NEOKINE-1 cDNA. This cDNA comprises sequences encoding the murine NEOKINE-1 protein (i.e., “the coding region”, from nucleotides 212-508), as well as 5′ untranslated sequences (nucleotides 1-211) and 3′ untranslated sequences (nucleotides 509-1656). Alternatively, the nucleic acid molecule can comprise only the coding region of SEQ ID NO:4 (e.g., nucleotides 212-508, corresponding to SEQ ID NO:6).

In another preferred embodiment, an isolated nucleic acid molecule of the invention comprises the nucleotide sequence shown in SEQ ID NO:7. The sequence of SEQ ID NO:7 corresponds to a rat NEOKINE-1 cDNA. This cDNA comprises sequences encoding at least 79 amino acid residues of the rat NEOKINE-1 protein (i.e., “the coding region”, from nucleotides 1-234), as well as 3′ untranslated sequences (nucleotides 235-1372). Alternatively, the nucleic acid molecule can comprise only the coding region of SEQ ID NO:7 (e.g., nucleotides 235-1372, corresponding to SEQ ID NO:9).

In another preferred embodiment, an isolated nucleic acid molecule of the invention comprises the nucleotide sequence shown in SEQ ID NO:10. The sequence of SEQ ID NO:10 corresponds to a macaque NEOKINE-1 cDNA. This cDNA comprises sequences encoding at least 94 amino acid residues of the macaque NEOKINE-1 protein (i.e., “the coding region”, from nucleotides 3-284), as well as 3′ untranslated sequences (nucleotides 285-1458). Alternatively, the nucleic acid molecule can comprise only the coding region of SEQ ID NO:10 (e.g., nucleotides 285-1458, corresponding to SEQ ID NO:22).

In another preferred embodiment, an isolated nucleic acid molecule of the invention comprises a nucleic acid molecule which is a complement of the nucleotide sequence shown in SEQ ID NO:1, SEQ ID NO:4, SEQ ID NO:7, SEQ ID NO:10, or the nucleotide sequence of the DNA insert of the plasmid deposited with ATCC as Accession Number 98751, or a portion of any of these nucleotide sequences. A nucleic acid molecule which is complementary to the nucleotide sequence shown in SEQ ID NO:1, SEQ ID NO:4, SEQ ID NO:7, SEQ ID NO:10, or the nucleotide sequence of the DNA insert of the plasmid deposited with ATCC as Accession Number 98751, is one which is sufficiently complementary to the nucleotide sequence shown in SEQ ID NO:1, SEQ ID NO:4, SEQ ID NO:7, SEQ ID NO:10, or the nucleotide sequence of the DNA insert of the plasmid deposited with ATCC as Accession Number 98751, such that it can hybridize to the nucleotide sequence shown in SEQ ID NO:1, SEQ ID NO:4, SEQ ID NO:7, SEQ ID NO:10, or the nucleotide sequence of the DNA insert of the plasmid deposited with ATCC as Accession Number 98751, thereby forming a stable duplex.

In still another preferred embodiment, an isolated nucleic acid molecule of the present invention comprises a nucleotide sequence which is at least about 60-65%, preferably at least about 70-75%, more preferable at least about 80-85%, and even more preferably at least about 90-95%, 96-97%, 98-99% or more homologous to the nucleotide sequences shown in SEQ ID NO:1, SEQ ID NO:4, SEQ ID NO:7, SEQ ID NO:10, or the nucleotide sequence of the DNA insert of the plasmid deposited with ATCC as Accession Number 98751, or a portion of any of these nucleotide sequences.

Moreover, the nucleic acid molecule of the invention can comprise only a portion of the nucleic acid sequence of SEQ ID NO:1, SEQ ID NO:4, SEQ ID NO:7, SEQ ID NO:10, or the nucleotide sequence of the DNA insert of the plasmid deposited with ATCC as Accession Number 98751, for example a fragment which can be used as a probe or primer or a fragment encoding a biologically active portion of a NEOKINE protein. The nucleotide sequence determined from the cloning of the NEOKINE-1 genes allows for the generation of probes and primers designed for use in identifying and/or cloning other NEOKINE family members, as well as NEOKINE homologues from other species. The probe/primer typically comprises substantially purified oligonucleotide. The oligonucleotide typically comprises a region of nucleotide sequence that hybridizes under stringent conditions to at least about 12, preferably about 25, more preferably about 40, 50 or 75 consecutive nucleotides of a sense sequence of SEQ ID NO:1, SEQ ID NO:4, SEQ ID NO:7, SEQ ID NO:10, or the nucleotide sequence of the DNA insert of the plasmid deposited with ATCC as Accession Number 98751, of an anti-sense sequence of SEQ ID NO:1, SEQ ID NO:4, SEQ ID NO:7, SEQ ID NO:10, or the nucleotide sequence of the DNA insert of the plasmid deposited with ATCC as Accession Number 98751, or of a naturally occurring mutant of SEQ ID NO:1, SEQ ID NO:4, SEQ ID NO:7, SEQ ID NO:10, or the nucleotide sequence of the DNA insert of the plasmid deposited with ATCC as Accession Number 98751. In an exemplary embodiment, a nucleic acid molecule of the present invention comprises a nucleotide sequence which is greater that 500 nucleotides in length and hybridizes under stringent hybridization conditions to a nucleic acid molecule of SEQ ID NO:1, SEQ ID NO:4, SEQ ID NO:7, SEQ ID NO:10, or the nucleotide sequence of the DNA insert of the plasmid deposited with ATCC as Accession Number 98751.

Probes based on the NEOKINE nucleotide sequences can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a NEOKINE protein, such as by measuring a level of a NEOKINE-encoding nucleic acid in a sample of cells from a subject e.g., detecting NEOKINE mRNA levels or determining whether a genomic NEOKINE gene has been mutated or deleted.

A nucleic acid fragment encoding a “biologically active portion of a NEOKINE protein” can be prepared by isolating a portion of SEQ ID NO:1, SEQ ID NO:4, SEQ ID NO:7, SEQ ID NO:10, or the nucleotide sequence of the DNA insert of the plasmid deposited with ATCC as Accession Number 98751, which encodes a polypeptide having a NEOKINE biological activity (the biological activities of the NEOKINE proteins have previously been described), expressing the encoded portion of the NEOKINE protein (e.g., by recombinant expression in vitro) and assessing the activity of the encoded portion of the NEOKINE protein.

The invention further encompasses nucleic acid molecules that differ from the nucleotide sequence shown in SEQ ID NO:1, SEQ ID NO:4, SEQ ID NO:7, SEQ ID NO:10, or the nucleotide sequence of the DNA insert of the plasmid deposited with ATCC as Accession Number 98751 due to degeneracy of the genetic code and thus encode the same NEOKINE proteins as those encoded by the nucleotide sequence shown in SEQ ID NO:1, SEQ ID NO:4, SEQ ID NO:7, SEQ ID NO:10, or the nucleotide sequence of the DNA insert of the plasmid deposited with ATCC as Accession Number 98751. In another embodiment, an isolated nucleic acid molecule of the invention has a nucleotide sequence encoding a protein having an amino acid sequence shown in SEQ ID NO:2, SEQ ID NO: 5, SEQ ID NO:8, or SEQ ID NO:11.

In addition to the NEOKINE nucleotide sequences shown in SEQ ID NO:1, SEQ ID NO:4, SEQ ID NO:7, SEQ ID NO:10, or the nucleotide sequence of the DNA insert of the plasmid deposited with ATCC as Accession Number 98751, it will be appreciated by those skilled in the art that DNA sequence polymorphisms that lead to changes in the amino acid sequences of the NEOKINE proteins may exist within a population (e.g., the human population). Such genetic polymorphism in the NEOKINE genes may exist among individuals within a population due to natural allelic variation. As used herein, the terms “gene” and “recombinant gene” refer to nucleic acid molecules comprising an open reading frame encoding a NEOKINE protein, preferably a mammalian NEOKINE protein. Such natural allelic variations can typically result in 1-5% variance in the nucleotide sequence of a NEOKINE gene. Any and all such nucleotide variations and resulting amino acid polymorphisms in NEOKINE genes that are the result of natural allelic variation and that do not alter the functional activity of a NEOKINE protein are intended to be within the scope of the invention.

Moreover, nucleic acid molecules encoding other NEOKINE family members (e.g., NEOKINE-2), and thus which have a nucleotide sequence which differs from the NEOKINE-1 sequences of SEQ ID NO:1, SEQ ID NO:4, SEQ ID NO:7, SEQ ID NO:10, or the nucleotide sequence of the DNA insert of the plasmid deposited with ATCC as Accession Number 98751 are intended to be within the scope of the invention. For example, a NEOKINE-2 cDNA can be identified based on the nucleotide sequence of human NEOKINE-1. Moreover, nucleic acid molecules encoding NEOKINE proteins from different species, and thus which have a nucleotide sequence which differs from the NEOKINE sequences of SEQ ID NO:1, SEQ ID NO:4, SEQ ID NO:7, SEQ ID NO:10, or the nucleotide sequence of the DNA insert of the plasmid deposited with ATCC as Accession Number 98751 are intended to be within the scope of the invention. For example, a Xenopus NEOKINE cDNA can be identified based on the nucleotide sequence of a human NEOKINE.

Nucleic acid molecules corresponding to natural allelic variants and homologues of the NEOKINE cDNAs of the invention can be isolated based on their homology to the NEOKINE nucleic acids disclosed herein using the cDNAs disclosed herein, or a portion thereof, as a hybridization probe according to standard hybridization techniques under stringent hybridization conditions.

Accordingly, in another embodiment, an isolated nucleic acid molecule of the invention is at least 15 nucleotides in length and hybridizes under stringent conditions to the nucleic acid molecule comprising the nucleotide sequence of SEQ ID NO:1, SEQ ID NO:4, SEQ ID NO:7, SEQ ID NO:10, or the nucleotide sequence of the DNA insert of the plasmid deposited with ATCC as Accession Number 98751. In other embodiment, the nucleic acid is at least 30, 50, 100, 250 or 500 nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 70%, more preferably at least about 80%, even more preferably at least about 85% or 90% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50° C., preferably 55° C., and more preferably 60° C. or 65° C. Preferably, an isolated nucleic acid molecule of the invention that hybridizes under stringent conditions to the sequence of SEQ ID NO:1, SEQ ID NO:4, SEQ ID NO:7, or SEQ ID NO:10, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the NEOKINE sequences that may exist in the population, the skilled artisan will further appreciate that changes can be introduced by mutation into the nucleotide sequences of SEQ ID NO:1, SEQ ID NO:4, SEQ ID NO:7, SEQ ID NO:10, or the nucleotide sequence of the DNA insert of the plasmid deposited with ATCC as Accession Number 98751, thereby leading to changes in the amino acid sequence of the encoded NEOKINE proteins, without altering the functional ability of the NEOKINE proteins. For example, nucleotide substitutions leading to amino acid substitutions at “non-essential” amino acid residues can be made in the sequence of SEQ ID NO:1, SEQ ID NO:4, SEQ ID NO:7, SEQ ID NO:10, or the nucleotide sequence of the DNA insert of the plasmid deposited with ATCC as Accession Number 98751. A “non-essential” amino acid residue is a residue that can be altered from the wild-type sequence of NEOKINE (e.g., the sequence of SEQ ID NO:2, SEQ ID NO:5, SEQ ID NO:8, or SEQ ID NO:11) without altering the biological activity, whereas an “essential” amino acid residue is required for biological activity. For example, amino acid residues that are conserved among the NEOKINE proteins of the present invention, are predicted to be particularly unamenable to alteration (e.g., the four conserved cycteines). Moreover, amino acid residues that are defined by the NEOKINE CXC motif are particularly unamenable to alteration. Furthermore, additional amino acid residues that are conserved between the NEOKINE proteins of the present invention as depicted in FIG. 5 are not likely to be amenable to alteration.

Accordingly, another aspect of the invention pertains to nucleic acid molecules encoding NEOKINE proteins that contain changes in amino acid residues that are not essential for activity. Such NEOKINE proteins differ in amino acid sequence from SEQ ID NO:2, SEQ ID NO:5, SEQ ID NO:8, or SEQ ID NO:11 yet retain biological activity. In one embodiment, the isolated nucleic acid molecule comprises a nucleotide sequence encoding a protein, wherein the protein comprises an amino acid sequence at least about 60% homologous to the amino acid sequence of SEQ ID NO:2, SEQ ID NO:5, SEQ ID NO:8, or SEQ ID NO:11. Preferably, the protein encoded by the nucleic acid molecule is at least about 65-70% homologous to SEQ ID NO:2, SEQ ID NO:5, SEQ ID NO:8, or SEQ ID NO:11, more preferably at least about 75-80% homologous to SEQ ID NO:2, SEQ ID NO:5, SEQ ID NO:8, or SEQ ID NO:11, even more preferably at least about 85-90% homologous to SEQ ID NO:2, SEQ ID NO:5, SEQ ID NO:8, or SEQ ID NO:11, and most preferably at least about 95% homologous to SEQ ID NO:2, SEQ ID NO:5, SEQ ID NO:8, or SEQ ID NO:11.

An isolated nucleic acid molecule encoding a NEOKINE protein homologous to the protein of SEQ ID NO:2, SEQ ID NO:5, SEQ ID NO:8, or SEQ ID NO:11 can be created by introducing one or more nucleotide substitutions, additions or deletions into the nucleotide sequence of SEQ ID NO:1, SEQ ID NO:4, SEQ ID NO:7, SEQ ID NO:10, or the nucleotide sequence of the DNA insert of the plasmid deposited with ATCC as Accession Number 98751, such that one or more amino acid substitutions, additions or deletions are introduced into the encoded protein. Mutations can be introduced into SEQ ID NO:1, SEQ ID NO:4, SEQ ID NO:7, SEQ ID NO:10, or the nucleotide sequence of the DNA insert of the plasmid deposited with ATCC as Accession Number 98751 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a NEOKINE protein is preferably replaced with another amino acid residue from the same side chain family. Alternatively, in another embodiment, mutations can be introduced randomly along all or part of a NEOKINE coding sequence, such as by saturation mutagenesis, and the resultant mutants can be screened for NEOKINE biological activity to identify mutants that retain activity. Following mutagenesis of SEQ ID NO:1, SEQ ID NO:4, SEQ ID NO:7, SEQ ID NO:10, or the nucleotide sequence of the DNA insert of the plasmid deposited with ATCC as Accession Number 98751, the encoded protein can be expressed recombinantly and the activity of the protein can be determined.

In a preferred embodiment, a mutant NEOKINE protein can be assayed for (1) modulation of cellular signal transduction, either in vitro or in vivo; (2) regulation of gene transcription in a cell expressing a NEOKINE receptor (e.g., RDC1) or receptor which is specific for another chemokine; (3) regulation of gene transcription in a cell expressing a NEOKINE receptor or receptor which is specific for another chemokine, wherein said cell is involved in angiogenesis or inflammation; (4) regulation of angiogenesis; (5) regulation of angiogenesis, wherein said regulation comprises inhibibition of angiogenesis; (6) regulation of angiogenesis, wherein said regulation comprises maintenance of angiostasis; (7) regulation of inflammation; and (8) regulation of inflammation, wherein said regulation comprises inhibition of chemoattraction (e.g., neutrophil chemoattraction).

In addition to the nucleic acid molecules encoding NEOKINE proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire NEOKINE coding strand, or to only a portion thereof. In one embodiment, an antisense nucleic acid molecule is antisense to a “coding region” of the coding strand of a nucleotide sequence encoding NEOKINE. The term “coding region” refers to the region of the nucleotide sequence comprising codons which are translated into amino acid residues (e.g., the coding region of human NEOKINE-1 corresponds to SEQ ID NO:3, the coding region of murine NEOKINE-1 corresponds to SEQ ID NO:6, and a coding region of rat NEOKINE-1 corresponds to SEQ ID NO:9). In another embodiment, the antisense nucleic acid molecule is antisense to a “noncoding region” of the coding strand of a nucleotide sequence encoding NEOKINE. The term “noncoding region” refers to 5′ and 3′ sequences which flank the coding region that are not translated into amino acids (i.e., also referred to as 5′ and 3′ untranslated regions). **

Given the coding strand sequences encoding NEOKINE disclosed herein (e.g., SEQ ID NO:3, SEQ ID NO:6, or SEQ ID NO:9), antisense nucleic acids of the invention can be designed according to the rules of Watson and Crick base pairing. The antisense nucleic acid molecule can be complementary to the entire coding region of NEOKINE mRNA, but more preferably is an oligonucleotide which is antisense to only a portion of the coding or noncoding region of NEOKINE mRNA. For example, the antisense oligonucleotide can be complementary to the region surrounding the translation start site of NEOKINE mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NEOKINE protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave NEOKINE mRNA transcripts to thereby inhibit translation of NEOKINE mRNA. A ribozyme having specificity for a NEOKINE-encoding nucleic acid can be designed based upon the nucleotide sequence of a NEOKINE-1 cDNA disclosed herein (i.e., SEQ ID NO:1, SEQ ID NO:4, SEQ ID NO:7, SEQ ID NO:10, or the nucleotide sequence of the DNA insert of the plasmid deposited with ATCC as Accession Number 98751). For example, a derivative of a Tetrahymena L-19 IVS RNA can be constructed in which the nucleotide sequence of the active site is complementary to the nucleotide sequence to be cleaved in a NEOKINE-encoding mRNA. See, e.g., Cech et al. U.S. Pat. No. 4,987,071; and Cech et al. U.S. Pat. No. 5,116,742. Alternatively, NEOKINE mRNA can be used to select a catalytic RNA having a specific ribonuclease activity from a pool of RNA molecules. See, e.g., Bartel and Szostak (1993) Science 261:1411-1418.

Alternatively, NEOKINE gene expression can be inhibited by targeting nucleotide sequences complementary to the regulatory region of the NEOKINE (e.g., the NEOKINE promoter and/or enhancers) to form triple helical structures that prevent transcription of the NEOKINE gene in target cells. See generally, Helene (1991) Anticancer Drug Des. 6:569-84; Helene et al. (1992) Ann. N.Y. Acad. Sci. 660:27-36; and Maher (1992) Bioassays 14(12):807-15.

In yet another embodiment, the NEOKINE nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup et al. (1996) Bioorganic & Medicinal Chemistry 4:5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) supra; Perry-O'Keefe et al. PNAS 93:14670-675.

PNAs of NEOKINE nucleic acid molecules can be used therapeutic and diagnostic applications. For example, PNAs can be used as antisense or antigene agents for sequence-specific modulation of gene expression by, for example, inducing transcription or translation arrest or inhibiting replication. PNAs of NEOKINE nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup et al. supra)); or as probes or primers for DNA sequencing or hybridization (Hyrup et al. supra; Perry-O'Keefe supra).

In another embodiment, PNAs of NEOKINE can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of NEOKINE nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup et al. supra). The synthesis of PNA-DNA chimeras can be performed as described in Hyrup et al. supra and Finn et al. (1996) Nucleic Acids Res. 24: 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag et al. (1989) Nucleic Acid Res. 17: 5973-88). PNA monomers are then coupled in a stepwise manner to produce a chimeric molecule with a 5′ PNA segment and a 3′ DNA segment (Finn et al. supra). Alternatively, chimeric moleclues can be synthesized with a 5′ DNA segment and a 3′ PNA segment (Peterser et al. (1975) Bioorganic Med. Chem. Lett. 5:1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. US. 86:6553-6556; Lemaitre et al. (1987) Proc. Natl. Acad. Sci. USA 84:648-652; PCT Publication No. W088/09810, published Dec. 15, 1988) or the blood-brain barrier (see, e.g., PCT Publication No. W089/10134, published Apr. 25, 1988). In addition, oligonucleotides can be modified with hybridization-triggered cleavage agents (See, e.g., Krol et al. (1988) BioTechniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

II. Isolated NEOKINE Proteins and Anti-NEOKINE Antibodies

One aspect of the invention pertains to isolated NEOKINE proteins, and biologically active portions thereof, as well as polypeptide fragments suitable for use as immunogens to raise anti-NEOKINE antibodies. In one embodiment, native NEOKINE proteins can be isolated from cells or tissue sources by an appropriate purification scheme using standard protein purification techniques. In another embodiment, NEOKINE proteins are produced by recombinant DNA techniques. Alternative to recombinant expression, a NEOKINE protein or polypeptide can be synthesized chemically using standard peptide synthesis techniques.

An “isolated” or “purified” protein or biologically active portion thereof is substantially free of cellular material or other contaminating proteins from the cell or tissue source from which the NEOKINE protein is derived, or substantially free from chemical precursors or other chemicals when chemically synthesized. The language “substantially free of cellular material” includes preparations of NEOKINE protein in which the protein is separated from cellular components of the cells from which it is isolated or recombinantly produced. In one embodiment, the language “substantially free of cellular material” includes preparations of NEOKINE protein having less than about 30% (by dry weight) of non-NEOKINE protein (also referred to herein as a “contaminating protein”), more preferably less than about 20% of non-NEOKINE protein, still more preferably less than about 10% of non-NEOKINE protein, and most preferably less than about 5% non-NEOKINE protein. When the NEOKINE protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of NEOKINE protein in which the protein is separated from chemical precursors or other chemicals which are involved in the synthesis of the protein. In one embodiment, the language “substantially free of chemical precursors or other chemicals” includes preparations of NEOKINE protein having less than about 30% (by dry weight) of chemical precursors or non-NEOKINE chemicals, more preferably less than about 20% chemical precursors or non-NEOKINE chemicals, still more preferably less than about 10% chemical precursors or non-NEOKINE chemicals, and most preferably less than about 5% chemical precursors or non-NEOKINE chemicals.

Biologically active portions of a NEOKINE protein include peptides comprising amino acid sequences sufficiently homologous to or derived from the amino acid sequence of the NEOKINE protein, e.g., the amino acid sequence shown in SEQ ID NO:2, SEQ ID NO:5, SEQ ID NO:8, or SEQ ID NO:11, which include less amino acids than the full length NEOKINE proteins, and exhibit at least one activity of a NEOKINE protein. Typically, biologically active portions comprise a domain or motif with at least one activity of the NEOKINE protein. A biologically active portion of a NEOKINE protein can be a polypeptide which is, for example, 10, 25, 50, 100 or more amino acids in length.

In one embodiment, a biologically active portion of a NEOKINE protein comprises at least a NEOKINE CXC signature motif. In another embodiment, a biologically active portion of a NEOKINE protein comprises at least a signal sequence. In another embodiment, a biologically active portion of a NEOKINE protein comprises a NEOKINE amino acid sequence lacking a signal sequence (e.g., a mature NEOKINE protein).

It is to be understood that a preferred biologically active portion of a NEOKINE protein of the present invention may contain at least one of the above-identified structural domains. A more preferred biologically active portion of a NEOKINE protein may contain at least two of the above-identified structural domains. Moreover, other biologically active portions, in which other regions of the protein are deleted, can be prepared by recombinant techniques and evaluated for one or more of the functional activities of a native NEOKINE protein.

In a preferred embodiment, the NEOKINE protein has an amino acid sequence shown in SEQ ID NO:2, SEQ ID NO:5, SEQ ID NO:8, or SEQ ID NO:11. In other embodiments, the NEOKINE protein is substantially homologous to SEQ ID NO:2, SEQ ID NO:5, SEQ ID NO:8, or SEQ ID NO:1, and retains the functional activity of the protein of SEQ ID NO:2, SEQ ID NO:5, SEQ ID NO:8, or SEQ ID NO:11, yet differs in amino acid sequence due to natural allelic variation or mutagenesis, as described in detail in subsection I above. Accordingly, in another embodiment, the NEOKINE protein is a protein which comprises an amino acid sequence at least about 60% homologous to the amino acid sequence of SEQ ID NO:2, SEQ ID NO:5, SEQ ID NO:8, or SEQ ID NO:11 and retains the functional activity of the NEOKINE proteins of SEQ ID NO:2, SEQ ID NO:5, SEQ ID NO:8, or SEQ ID NO:11, respectively. Preferably, the protein is at least about 70% homologous to SEQ ID NO:2, SEQ ID NO:5, SEQ ID NO:8, or SEQ ID NO:11, more preferably at least about 80% homologous to SEQ ID NO:2, SEQ ID NO:5, SEQ ID NO:8, or SEQ ID NO:11, even more preferably at least about 90% homologous to SEQ ID NO:2, SEQ ID NO:5, SEQ ID NO:8, or SEQ ID NO:11, and most preferably at least about 95% or more homologous to SEQ ID NO:2, SEQ ID NO:5,SEQ ID NO:8, or SEQ ID NO:11.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the NEOKINE amino acid sequence of SEQ ID NO:2 having 99 amino acid residues, at least 30, preferably at least 40, more preferably at least 50, even more preferably at least 59, and even more preferably at least 69, 79, or 89 are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions×100).

The comparison of sequences and determination of percent homology between two sequences can be accomplished using a mathematical algorithim. A preferred, non-limiting example of a mathematical algorithim utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs (version 2.0) of Altschul et al. (1990) J. Mol. Biol. 215:403-10. BLAST nucleotide searches can be performed performed with the NBLAST program, score=100, wordlength=12 to obtain nucleotide sequences homologous to NEOKINE nucleic acid molecules of the invention. BLAST protein searches can be performed with the XBLAST program, score=50, wordlength=3 to obtain amino acid sequences homologous to NEOKINE protein molecules of the invention. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithim utilized for the comparison of sequences is the algorithm of Myers and Miller (1989) CABIOS. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The invention also provides NEOKINE chimeric or fusion proteins. As used herein, a NEOKINE “chimeric protein” or “fusion protein” comprises a NEOKINE polypeptide operatively linked to a non-NEOKINE polypeptide. A “NEOKINE polypeptide” refers to a polypeptide having an amino acid sequence corresponding to NEOKINE, whereas a “non-NEOKINE polypeptide” refers to a polypeptide having an amino acid sequence corresponding to a protein which is not substantially homologous to the NEOKINE protein, e.g., a protein which is different from the NEOKINE protein and which is derived from the same or a different organism. Within a NEOKINE fusion protein the NEOKINE polypeptide can correspond to all or a portion of a NEOKINE protein. In a preferred embodiment, a NEOKINE fusion protein comprises at least one biologically active portion of a NEOKINE protein. In another preferred embodiment, a NEOKINE fusion protein comprises at least two biologically active portions of a NEOKINE protein. Within the fusion protein, the term “operatively linked” is intended to indicate that the NEOKINE polypeptide and the non-NEOKINE polypeptide are fused in-frame to each other. The non-NEOKINE polypeptide can be fused to the N-terminus or C-terminus of the NEOKINE polypeptide.

For example, in one embodiment, the fusion protein is a GST-NEOKINE fusion protein in which the NEOKINE sequences are fused to the C-terminus of the GST sequences. Such fusion proteins can facilitate the purification of recombinant NEOKINE.

In another embodiment, the fusion protein is a NEOKINE protein containing a heterologous signal sequence at its N-terninus. For example, the native NEOKINE signal sequence (i.e, about amino acids 1 to 22 of SEQ ID NO:2) can be removed and replaced with a signal sequence from another protein. In certain host cells (e.g., mammalian host cells), expression and/or secretion of NEOKINE can be increased through use of a heterologous signal sequence.

In yet another embodiment, the fusion protein is a NEOKINE-immunoglobulin fusion protein in which the NEOKINE sequences comprising primarily the mature NEOKINE protein sequences are fused to sequences derived from a member of the immunoglobulin protein family. Soluble derivatives have also been made of cell surface glycoproteins in the immunoglobulin gene superfamily consisting of an extracellular domain of the cell surface glycoprotein fused to an immunoglobulin constant (Fc) region (see e.g., Capon et al. (1989) Nature 337:525-531 and Capon U.S. Pat. Nos. 5,116,964 and 5,428,130 [CD4-IgG1 constructs]; Linsley et al. (1991) J. Exp. Med. 173:721-730 [a CD28-IgG1 construct and a B7-1-IgG1 construct]; and Linsley et al. (1991) J. Exp. Med. 174:561-569 and U.S. Pat. No. 5,434,131[a CTLA4-IgG1]). Such fusion proteins have proven useful for modulating receptor-ligand interactions. Soluble derivatives of cell surface proteins of the tumor necrosis factor receptor (TNFR) superfamily proteins have been made consisting of an extracellular domain of the cell surface receptor fused to an immunoglobulin constant (Fc) region (See for example Moreland et al. (1997) N. Engl. J. Med. 337:141-147; van der Poll et al. (1997) Blood 89:3727-3734; and Ammann et al. (1997) J. Clin. Invest. 99:1699-1703.)

In yet another embodiment, the fusion protein comprises NEOKINE sequences (e.g., the NEOKINE CXC signature motif) fused to sequences form other CXC cytokines. For example, NEOKINE sequences C-terninal to and including the first conserved cysteine residues can be fused to N-terminal sequences of other non-NEOKINE chemokines (e.g., comprising from the N-terminal amino acid residue to the amino acid residue N-terminal to the first conserved cysteine). The NEOKINE fusion proteins of the invention can be incorporated into pharmaceutical compositions and administered to a subject to inhibit an interaction between a NEOKINE and a NEOKINE receptor on the surface of a cell, to thereby suppress NEOKINE-mediated signal transduction in vivo. The NEOKINE fusion proteins can be used to affect the bioavailability of a NEOKINE cognate receptor. The NEOKINE fusion proteins of the invention can further be used to inhibit an interaction between a chemokine other than a NEOKINE of the present invention and a chemokine receptor on the surface of a cell, to thereby suppress chemokine-mediated signal transduction in vivo. The NEOKINE fusion proteins can be used to affect the bioavailability of a NEOKINE cognate receptor. Use of NEOKINE fusion proteins may be useful therapeutically for the treatment of inflammation (e.g., kidney inflammation), as well for regulating angiogenesis (e.g., promoting or inhibiting angiogenesis or maintaining angiostasis). Moreover, the NEOKINE-fusion proteins of the invention can be used as immunogens to produce anti-NEOKINE antibodies in a subject, to purify NEOKINE ligands and in screening assays to identify molecules which inhibit the interaction of NEOKINE with a NEOKINE ligand.

Preferably, a NEOKINE chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A NEOKINE-encoding nucleic acid can be cloned into such an expression vector such that the fusion moiety is linked in-frame to the NEOKINE protein.

The present invention also pertains to variants of the NEOKINE proteins which function as either NEOKINE agonists (mimetics) or as NEOKINE antagonists. Variants of the NEOKINE proteins can be generated by mutagenesis, e.g., discrete point mutation or truncation of a NEOKINE protein. An agonist of the NEOKINE proteins can retain substantially the same, or a subset, of the biological activities of the naturally occurring form of a NEOKINE protein. An antagonist of a NEOKINE protein can inhibit one or more of the activities of the naturally occurring form of the NEOKINE protein by, for example, competitively binding to a NEOKINE or non-NEOKINE receptor. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NEOKINE protein.

In one embodiment, variants of a NEOKINE protein which function as either NEOKINE agonists (mimetics) or as NEOKINE antagonists can be identified by screening combinatorial libraries of mutants, e.g., truncation mutants, of a NEOKINE protein for NEOKINE protein agonist or antagonist activity. In one embodiment, a variegated library of NEOKINE variants is generated by combinatorial mutagenesis at the nucleic acid level and is encoded by a variegated gene library. A variegated library of NEOKINE variants can be produced by, for example, enzymatically ligating a mixture of synthetic oligonucleotides into gene sequences such that a degenerate set of potential NEOKIME sequences is expressible as individual polypeptides, or alternatively, as a set of larger fusion proteins (e.g., for phage display) containing the set of NEOKINE sequences therein. There are a variety of methods which can be used to produce libraries of potential NEOKINE variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NEOKINE sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a NEOKINE protein coding sequence can be used to generate a variegated population of NEOKINE fragments for screening and subsequent selection of variants of a NEOKINE protein. In one embodiment, a library of coding sequence fragments can be generated by treating a double stranded PCR fragment of a NEOKINE coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S I nuclease, and ligating the resulting fragment library into an expression vector. By this method, an expression library can be derived which encodes N-terminal, C-terminal and internal fragments of various sizes of the NEOKINE protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NEOKINE proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique which enhances the frequency of functional mutants in the libraries, can be used in combination with the screening assays to identify NEOKINE variants (Arkin and Yourvan (1992) PNAS 89:7811-7815; Delgrave et al. (1993) Protein Engineering 6(3):327-331).

In one embodiment, cell based assays can be exploited to analyze a variegated NEOKINE library. For example, a library of expression vectors can be transfected into a cell line which ordinarily responds to a particular ligand in a NEOKINE-dependent manner. The transfected cells are then contacted with the ligand and the effect of expression of the mutant on signaling by the ligand can be detected, e.g., by measuring any of a number of inflammatory or angiogenic responses. Plasmid DNA can then be recovered from the cells which score for inhibition, or alternatively, potentiation of ligand induction, and the individual clones further characterized.

An isolated NEOKINE protein, or a portion or fragment thereof, can be used as an immunogen to generate antibodies that bind NEOKINE using standard techniques for polyclonal and monoclonal antibody preparation. A full-length NEOKINE protein can be used or, alternatively, the invention provides antigenic peptide fragments of NEOKINE for use as immunogens. The antigenic peptide of NEOKINE comprises at least 8 amino acid residues of the amino acid sequence shown in SEQ ID NO:2, SEQ ID NO:5, SEQ ID NO:8, or SEQ ID NO:11 and encompasses an epitope of NEOKINE such that an antibody raised against the peptide forms a specific immune complex with NEOKINE. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of NEOKINE that are located on the surface of the protein, e.g., hydrophilic regions.

A NEOKINE immunogen typically is used to prepare antibodies by immunizing a suitable subject, (e.g., rabbit, goat, mouse or other mammal) with the immunogen. An appropriate immunogenic preparation can contain, for example, recombinantly expressed NEOKINE protein or a chemically synthesized NEOKINE polypeptide. The preparation can further include an adjuvant, such as Freund's complete or incomplete adjuvant, or similar immunostimulatory agent. Immunization of a suitable subject with an immunogenic NEOKINE preparation induces a polyclonal anti-NEOKINE antibody response.

Accordingly, another aspect of the invention pertains to anti-NEOKINE antibodies. The term “antibody” as used herein refers to immunoglobulin molecules and immunologically active portions of immunoglobulin molecules, i.e., molecules that contain an antigen binding site which specifically binds (immunoreacts with) an antigen, such as NEOKINE. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind NEOKINE. The term “monoclonal antibody” or “monoclonal antibody composition”, as used herein, refers to a population of antibody molecules that contain only one species of an antigen binding site capable of immunoreacting with a particular epitope of NEOKINE. A monoclonal antibody composition thus typically displays a single binding affinity for a particular NEOKINE protein with which it immunoreacts.

Polyclonal anti-NEOKINE antibodies can be prepared as described above by immunizing a suitable subject with a NEOKINE immunogen. The anti-NEOKINE antibody titer in the immunized subject can be monitored over time by standard techniques, such as with an enzyme linked immunosorbent assay (ELISA) using immobilized NEOKINE. If desired, the antibody molecules directed against NEOKINE can be isolated from the mammal (e.g., from the blood) and further purified by well known techniques, such as protein A chromatography to obtain the IgG fraction. At an appropriate time after immunization, e.g., when the anti-NEOKINE antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a NEOKINE immunogen as described above, and the culture supernatants of the resulting hybridoma cells are screened to identify a hybridoma producing a monoclonal antibody that binds NEOKINE.

Any of the many well known protocols used for fusing lymphocytes and immortalized cell lines can be applied for the purpose of generating an anti-NEOKINE monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind NEOKINE, e.g., using a standard ELISA assay.

Alternative to preparing monoclonal antibody-secreting hybridomas, a monoclonal anti-NEOKINE antibody can be identified and isolated by screening a recombinant combinatorial immunoglobulin library (e.g., an antibody phage display library) with NEOKINE to thereby isolate immunoglobulin library members that bind NEOKINE. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:4133-4137; Barbas et al. (1991) PNAS 88:7978-7982; and McCafferty et al. Nature (1990) 348:552-554.

Additionally, recombinant anti-NEOKINE antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. 5,225,539; Jones et al. (1986) Nature 321:552-525; Verhoeyan et al. (1988) Science 239:1534; and Beidler et al. (1988) J. Immunol. 141:4053-4060.

An anti-NEOKINE antibody (e.g., monoclonal antibody) can be used to isolate NEOKINE by standard techniques, such as affinity chromatography or immunoprecipitation. An anti-NEOKINE antibody can facilitate the purification of natural NEOKINE from cells and of recombinantly produced NEOKINE expressed in host cells. Moreover, an anti-NEOKINE antibody can be used to detect NEOKINE protein (e.g., in a cellular lysate or cell supernatant) in order to evaluate the abundance and pattern of expression of the NEOKINE protein. Anti-NEOKINE antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, preferably expression vectors, containing a nucleic acid encoding a NEOKINE protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NEOKINE proteins, mutant forms of NEOKINE proteins, fusion proteins, etc.).

The recombinant expression vectors of the invention can be designed for expression of NEOKINE proetins in prokaryotic or eukaryotic cells. For example, NEOKINE proetins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:3140), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in NEOKINE activity assays, in NEOKINE ligand binding (e.g., direct assays or competitive assays described in detail below), to generate antibodies specific for NEOKINE proteins, as examples. In a preferred embodiment, a NEOKINE fusion protein expressed in a retroviral expression vector of the present invention can be utilized to infect bone marrow cells which are subsequently transplanted into irradiated recipients. The pathology of the subject recipient is then examined after sufficient time has passed (e.g six (6) weeks).

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS 174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the NEOKINE expression vector is a yeast expression vector. Examples of vectors for expression in yeast S. cerivisae include pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, NEOKINE proteins can be expressed in insect cells using baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to NEOKINE mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a NEOKINE protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a NEOKINE protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a NEOKINE protein. Accordingly, the invention further provides methods for producing a NEOKINE protein using the host cells of the invention. In one embodiment, the method comprises culturing the host cell of invention (into which a recombinant expression vector encoding a NEOKINE protein has been introduced) in a suitable medium such that a NEOKINE protein is produced. In another embodiment, the method further comprises isolating a NEOKINE protein from the medium or the host cell.

The host cells of the invention can also be used to produce nonhuman transgenic animals. For example, in one embodiment, a host cell of the invention is a fertilized oocyte or an embryonic stem cell into which NEOKINE-coding sequences have been introduced. Such host cells can then be used to create non-human transgenic animals in which exogenous NEOKINE sequences have been introduced into their genome or homologous recombinant animals in which endogenous NEOKINE sequences have been altered. Such animals are useful for studying the function and/or activity of a NEOKINE and for identifying and/or evaluating modulators of NEOKINE activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NEOKINE gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a NEOKINE-encoding nucleic acid into the male pronuclei of a fertilized oocyte, e.g., by microinjection, retroviral infection, and allowing the oocyte to develop in a pseudopregnant female foster animal. The NEOKINE-1 cDNA sequence of SEQ ID NO:1 can be introduced as a transgene into the genome of a non-human animal. Alternatively, a nonhuman homologue of a human NEOKINE-1 gene, such as a mouse NEOKINE-1 gene (SEQ ID NO:4), a rat NEOKINE-1 gene (SEQ ID NO:7), or a macaque NEOKINE cDNA (SEQ ID NO:10), can be used as a transgene. Alternatively, a NEOKINE-1 gene homologue, such as a NEOKINE-2 gene can be isolated based on hybridization to the NEOKINE-1 cDNA sequences of SEQ ID NO:1, SEQ ID NO:4, SEQ ID NO:7, or SEQ ID NO:10 (described further in subsection I above) and used as a transgene. Intronic sequences and polyadenylation signals can also be included in the transgene to increase the efficiency of expression of the transgene. A tissue-specific regulatory sequence(s) can be operably linked to a NEOKINE transgene to direct expression of a NEOKINE protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a NEOKINE transgene in its genome and/or expression of NEOKINE mRNA in tissues or cells of the animals. A transgenic founder animal can then be used to breed additional animals carrying the transgene. Moreover, transgenic animals carrying a transgene encoding a NEOKINE protein can further be bred to other transgenic animals carrying other transgenes.

To create a homologous recombinant animal, a vector is prepared which contains at least a portion of a NEOKINE gene into which a deletion, addition or substitution has been introduced to thereby alter, e.g., functionally disrupt, the NEOKINE gene. The NEOKINE gene can be a human gene (e.g., the cDNA of SEQ ID NO:3), but more preferably, is a non-human homologue of a human NEOKINE gene (e.g., the cDNA of SEQ ID NO: 6, or SEQ ID NO:9). For example, a mouse NEOKINE gene of SEQ ID NO:6 can be used to construct a homologous recombination vector suitable for altering an endogenous NEOKINE gene in the mouse genome. In a preferred embodiment, the vector is designed such that, upon homologous recombination, the endogenous NEOKINE gene is functionally disrupted (i.e., no longer encodes a functional protein; also referred to as a “knock out” vector). Alternatively, the vector can be designed such that, upon homologous recombination, the endogenous NEOKINE gene is mutated or otherwise altered but still encodes functional protein (e.g., the upstream regulatory region can be altered to thereby alter the expression of the endogenous NEOKINE protein). In the homologous recombination vector, the altered portion of the NEOKINE gene is flanked at its 5′ and 3′ ends by additional nucleic acid sequene of the NEOKINE gene to allow for homologous recombination to occur between the exogenous NEOKINE gene carried by the vector and an endogenous NEOKINE gene in an embryonic stem cell. The additional flanking NEOKINE nucleic acid sequence is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced NEOKINE gene has homologously recombined with the endogenous NEOKINE gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Bers et al.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813. In brief, a cell, e.g., a somatic cell, from the transgenic animal can be isolated and induced to exit the growth cycle and enter G_{O} phase. The quiescent cell can then be fused, e.g., through the use of electrical pulses, to an enucleated oocyte from an animal of the same species from which the quiescent cell is isolated. The recontructed oocyte is then cultured such that it develops to morula or blastocyte and then transferred to pseudopregnant female foster animal. The offspring borne of this female foster animal will be a clone of the animal from which the cell, e.g., the somatic cell, is isolated.

IV. Pharmaceutical Compositions

The NEOKINE nucleic acid molecules, NEOKINE proteins, and anti-NEOKINE antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NEOKINE protein or anti-NEOKINE antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The nucleic acid molecules of the invention can be inserted into vectors and used as gene therapy vectors. Gene therapy vectors can be delivered to a subject by, for example, intravenous injection, local administration (see U.S. Pat. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic). As described herein, a NEOKINE protein of the invention has one or more of the following activities: (i) interaction of a NEOKINE protein with a membrane-bound NEOKINE receptor; (ii) interaction of a NEOKINE protein with a membrane-bound chemokine receptor; (iii) indirect interaction of a NEOKINE protein with an intracellular protein via a membrane-bound NEOKINE receptor; (iv) indirect interaction of a NEOKINE protein with an intracellular protein via a membrane-bound chemokine receptor; (v) complex formation between a soluble NEOKINE protein and a NEOKINE binding partner; (vi) inhibition of the interaction of chemokines (e.g., pro-inflammatory chemokines) by binding to their cognate receptors; (vii) inhibition of the binding of HIV to HIV co-receptors; and (vii) inhibition of the binding of HIV to HIV co-receptors wherein said binding induces subsequent infection of susceptible cells and can can thus be used in, for example, (1) modulation of cellular signal transduction, either in vitro or in vivo; (2) regulation of gene transcription in a cell expressing a NEOKINE receptor or a chemokine receptor; (3) regulation of gene transcription in a cell expressing a NEOKINE receptor or a chemokine receptor, wherein said cell is involved in angiogenesis or inflammation; (4) regulation of angiogenesis; (5) regulation of angiogenesis, wherein said regulation comprises inhibibition of angiogenesis; (6) regulation of angiogenesis, wherein said regulation comprises maintenance of angiostasis; (7) regulation of inflammation; and (8) regulation of inflammation, wherein said regulation comprises inhibition of chemoattraction (e.g., neutrophil chemoattraction), inhibition of inflammation, inhibition of inflammation by blocking the action of pro-inflammatory chemokines by binding to their cognate receptors, inhibition of psoriasis, suppression of immune rejection following skin graft, suppression of immune rejection following kidney transplant, inhibition of kidney inflammation in acute renal failure, inhibition of brain inflammation following stroke or ischaemia, or inhibition of brain inflammation following viral infection. The isolated nucleic acid molecules of the invention can be used, for example, to express NEOKINE protein (e.g., via a recombinant expression vector in a host cell in gene therapy applications), to detect NEOKINE mRNA (e.g., in a biological sample) or a genetic alteration in a NEOKINE gene, and to modulate NEOKINE activity, as described further below. The NEOKINE proteins can be used to treat disorders characterized by insufficient or excessive production of a non-NEOKINE chemokine or production of chemokine forms which have decreased or aberrant activity compared to wild type chemokines. In addition, the NEOKINE proteins can be used to screen drugs or compounds which modulate the NEOKINE activity as well as to treat disorders characterized by insufficient or excessive production of NEOKINE protein or production of NEOKINE protein forms which have decreased or aberrant activity compared to NEOKINE wild type protein. Moreover, the anti-NEOKINE antibodies of the invention can be used to detect and isolate NEOKINE proteins, regulate the bioavailability of NEOKINE proteins, and modulate NEOKINE activity.

A. Screening Assays:

The invention provides a method (also referred to herein as a “screening assay”) for identifying modulators, i.e., candidate or test compounds or agents (e.g., peptides, peptidomimetics, small molecules or other drugs) which bind to NEOKINE proteins, bind to NEOKINE receptors, have a stimulatory or inhibitory effect on, for example, NEOKINE expression, NEOKINE activity, or NEOKINE receptor activity (e.g., RDC1 activity), or have a stimulatory or inhibitory effect on, for example, the expression or activity of a non-NEOKINE chemokine or non-NEOKINE chemokine receptor. It will be appreciated by one of skill in the art that modulators identified by the screening assays defined herein (e.g., modulators of NEOKINE and/or NEOKINE receptor or RDC1) can be used in the prophylactic and therapeutic treatment of diseases and disorders associated with aberrant NEOKINE and/or NEOKINE receptor activity (e.g., proliferative disorders and diseases).

In one embodiment, the invention provides assays for screening candidate or test compounds which bind to or modulate the activity of a NEOKINE protein or polypeptide or biologically active portion thereof. In another embodiment, the invention provides assays for screening candidate or test compounds which bind to or modulate the activity of a NEOKINE receptor. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a NEOKINE receptor on the cell surface is contacted with a test compound and the ability of the test compound to bind to a NEOKINE receptor determined. The cell, for example, can be of mammalian origin or a yeast cell. The NEOKINE receptor can be heterologously expressed or over expressed in a host cell (e.g., a COS cell or fibroblastic cell, for example a HEK293 cell). Alternatively, an assay cell can be seleceted which endogenously expresses a NEOKINE receptor (e.g., RDC1), for example, a rat pancreatic acinar cell line, AR4-2J, a PC12 pheochromocytoma cell, a SK-N-MC neroblastoma cell, a MES-13 mesangial tumor cell, an astrocyte, or a neutrophil). (Hesen et al. (1998) Immunogenetics 47:364-370 and Law and Rosenzweig (1994) Biochem. Biophys. Res. Commun. 201:458-465). Yeast cells are also particularly amenable for use in screening assays for G-protein-coupled receptors as described, for example, in Pausch (1997) TIBTECH 15:487-494. Determining the ability of the test compound to bind to a NEOKINE receptor can be accomplished, for example, by coupling the test compound with a radioisotope or enzymatic label such that binding of the test compound to the NEOKINE receptor can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a test compound to interact with an NEOKINE receptor without the labeling of any of the interactants. For example, a microphysiometer can be used to detect the interaction of a test compound with an NEOKINE receptor without the labeling of either the test compound or the receptor. McConnell, H. M. et al. (1992) Science 257:1906-1912. As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between ligand and receptor.

In a preferred embodiment, the assay comprises contacting a cell which expresses an NEOKINE receptor on the cell surface with a NEOKINE protein or biologically-active portion thereof and a test compound, and determining the ability of the test compound to modulate binding of the NEOKINE protein or biologically-active portion thereof to the NEOKINE receptor. Determining the ability of the test compound to modulate binding of the NEOKINE protein or biologically-active portion thereof to the NEOKINE receptor can comprise determining the ability of the test compound to preferentially bind to the NEOKINE receptor as compared to the ability of NEOKINE, or a biologically active portion thereof, to bind to the receptor. Alternatively, determining the ability of the test compound to modulate binding of the NEOKINE protein or biologically-active portion thereof to the NEOKINE receptor can comprise determining a change in the binding of the NEOKINE protein or biologically-active portion thereof to the NEOKINE receptor (e.g., a change in the amount of binding in the presence of the test compound as compared to the absence of the test compound).

In another preferred embodiment, the assay comprises contacting a cell which expresses a receptor specific for another chemokine on the cell surface with an NEOKINE protein or biologically-active portion thereof and a test compound, and determining the ability of the test compound to interact with the receptor, wherein determining the ability of the test compound to interact with the receptor comprises determining the ability of the test compound to preferentially bind to the receptor as compared to the ability of the NEOKINE, or a biologically active portion thereof, to bind to the receptor. Alternatively, determining the ability of the test compound to interact with the NEOKINE receptor can comprise determining a change in the binding of the NEOKINE protein or biologically-active portion thereof to the NEOKINE receptor (e.g., a change in the amount of binding).

In another embodiment, an assay is a cell-based assay comprising contacting a cell expressing a NEOKINE target molecule (e.g. a NEOKINE receptor) with a test compound and determining the ability of the test compound to modulate (e.g. stimulate or inhibit) the activity of the NEOKINE target molecule. In yet another embodiment, an assay is a cell-based assay comprising contacting a cell expressing a NEOKINE receptor with a NEOKINE protein or biologically-active portion thereof and a test compound and determining the ability of the test compound to modulate the activity of the NEOKINE target molecule.

Determining the ability of the NEOKINE protein to bind to or interact with an NEOKINE target molecule can be accomplished by one of the methods described above for determining direct binding. The activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NEOKINE-responsive regulatory element operatively linked to a nucleic acid encoding a detectable marker, e.g., luciferase), or detecting a cellular response, for example, an angiogenic response or an inflammatory response.

In yet another embodiment, an assay of the present invention is a cell-free assay in which an NEOKINE protein or biologically active portion thereof is contacted with a test compound and the ability of the test compound to bind to the NEOKINE protein or biologically active portion thereof is determined. Binding of the test compound to the NEOKINE protein can be determined either directly or indirectly as described above. In a preferred embodiment, the assay includes contacting the NEOKINE protein or biologically active portion thereof with a known compound which binds NEOKINE to form an assay mixture, contacting the assay mixture with a test compound, and determining the ability of the test compound to interact with an NEOKINE protein, wherein determining the ability of the test compound to interact with an NEOKINE protein comprises determining the ability of the test compound to preferentially bind to NEOKINE or biologically active portion thereof as compared to the known compound.

In another embodiment, the assay is a cell-free assay in which an NEOKINE protein or biologically active portion thereof is contacted with a test compound and the ability of the test compound to modulate (e.g., stimulate or inhibit) the activity of the NEOKINE protein or biologically active portion thereof is determined. Determining the ability of the test compound to modulate the activity of an NEOKINE protein can be accomplished, for example, by determining the ability of the NEOKINE protein to bind to an NEOKINE target molecule by one of the methods described above for determining direct binding. Determining the ability of the NEOKINE protein to bind to an NEOKINE target molecule can also be accomplished using a technology such as real-time Biomolecular Interaction Analysis (BIA). Sjolander and Urbaniczky (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore™). Changes in the optical phenomenon of surface plasmon resonance (SPR) can be used as an indication of real-time reactions between biological molecules.

In an alternative embodiment, determining the ability of the test compound to modulate the activity of an NEOKINE protein can be accomplished by determining the ability of the NEOKINE protein to further modulate the activity of a downstream effector (e.g., an intracellular signaling molecule) of an NEOKINE target molecule (e.g., an NEOKINE receptor). For example, the catalytic/enzymatic activity of the effector molecule on an appropriate substrate can be determined as previously described.

In yet another embodiment, the cell-free assay involves contacting an NEOKINE protein or biologically active portion thereof with a known compound which binds the NEOKINE protein to form an assay mixture, contacting the assay mixture with a test compound, and determining the ability of the test compound to interact with the NEOKINE protein, wherein determining the ability of the test compound to interact with the NEOKINE protein comprises determining the ability of the NEOKINE protein to preferentially bind to or modulate the activity of an NEOKINE target molecule.

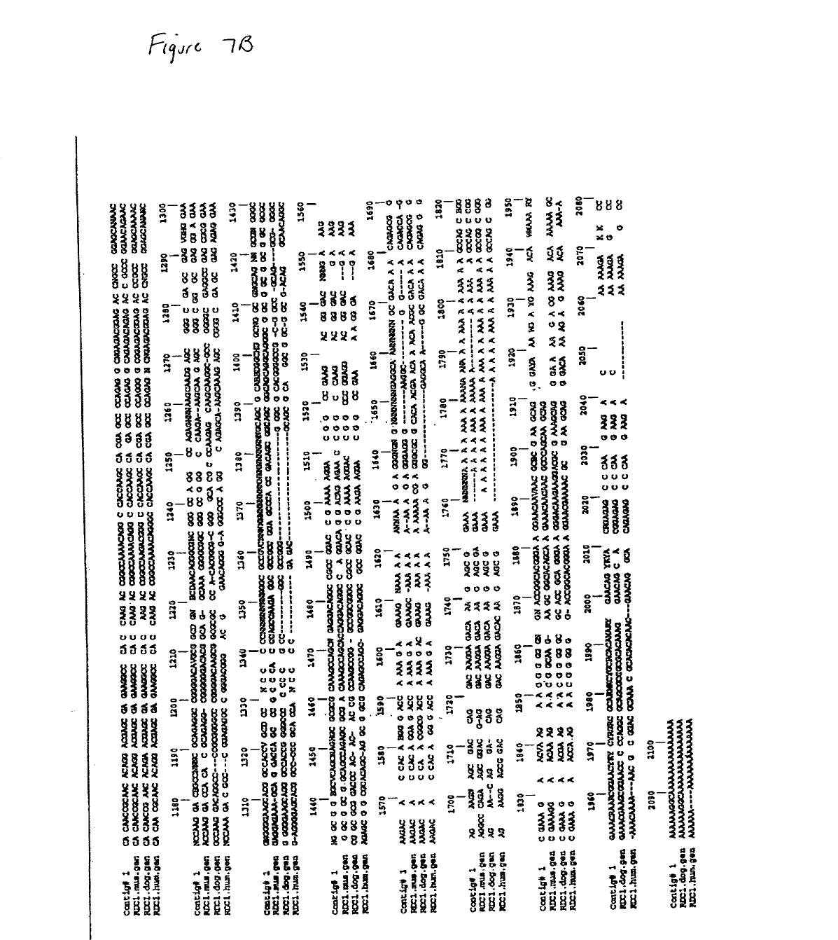

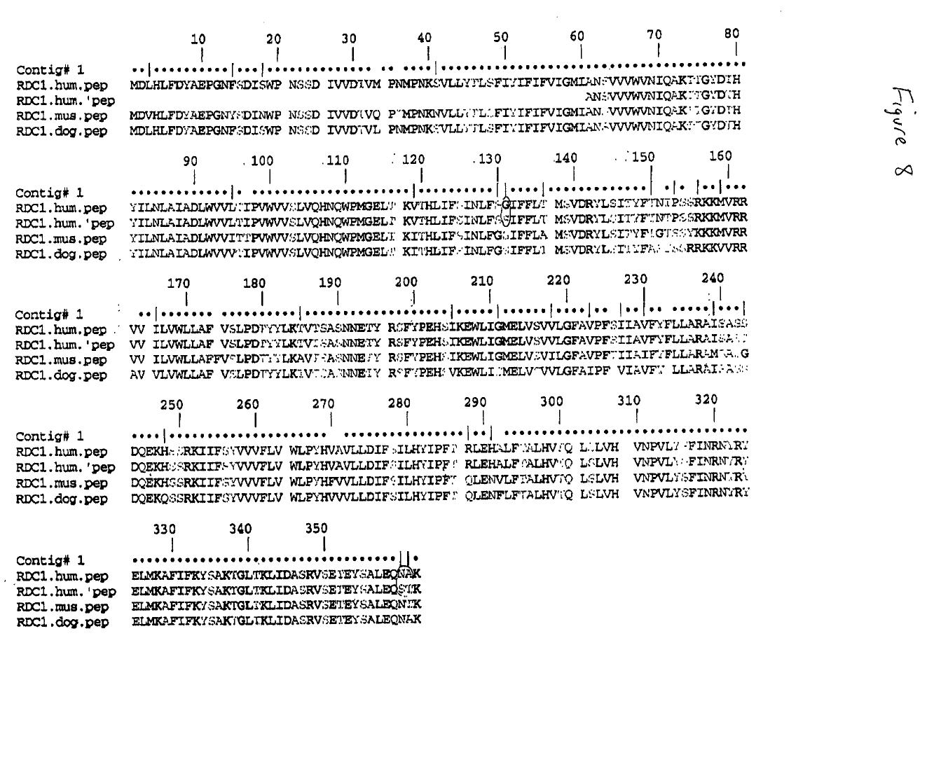

The assays of the present invention are based at least in part on the discovery that NEOKINE receptor is the previously identified orphan chemokine receptor, RDC1. The nucleic acid sequence of human (SEQ ID NO:15), murine (SEQ ID NO:17) and canine (SEQ ID NO:19) RDC1 are set forth in FIG. 7. The amino acid sequences of human (SEQ ID NO:16), murine (SEQ ID NO:18) and canine (SEQ ID NO:20) RDC1 are set forth in FIG. 8. Human, murine and canine RDC1 sequences can be further found at Accession Nos. U73141 & U67784, AF000236, and X14048, respectively. Accordingly, in one embodiment, the NEOKINE receptor has the amino acid set forth in any of SEQ ID NO:16, SEQ ID NO:18, or SEQ ID:NO 20. In another embodiment, the NEOKINE receptor is selected encoded by a nucleic acid molecule selected from the group consisting of SEQ ID NO:15, SEQ ID NO:17, or SEQ ID NO:19. In another embodiment the NEOKINE receptor is selected from the group consisting of a receptor having an amino acid sequence which is substantially homologous to the amino acid sequence of any of SEQ ID NO:16, SEQ ID NO:18, or SEQ ID NO:20; a receptor which is encoded by an isolated nucleic acid molecule which is substantially homologous to any of SEQ ID NO:15, SEQ ID NO:17, or SEQ ID NO:19; or a receptor which is encoded by an isolated nucleic acid molecule which hybridizes under stringent conditions to a nucleic acid molecule having any of SEQ ID NO:15, SEQ ID NO:17, or SEQ ID NO:17.

The cell-free assays of the present invention are amenable to use of both soluble and/or membrane-bound forms of isolated proteins (e.g. NEOKINE proteins or biologically active portions thereof or NEOKINE receptors). In the case of cell-free assays in which a membrane-bound form an isolated protein is used (e.g., a NEOKINE receptor) it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl═N,N-dimethyl-3-ammonio-1-propane sulfonate.

In more than one embodiment of the above assay methods of the present invention, it may be desirable to immobilize either NEOKINE or its target molecule to facilitate separation of complexed from uncomplexed forms of one or both of the proteins, as well as to accommodate automation of the assay. Binding of a test compound to a NEOKINE protein, or interaction of a NEOKINE protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/NEOKINE fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or NEOKINE protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of NEOKINE binding or activity determined using standard techniques.

Other techniques for immobilizing proteins on matrices can also be used in the screening assays of the invention. For example, either a NEOKINE protein or a NEOKINE target molecule can be immobilized utilizing conjugation of biotin and streptavidin. Biotinylated NEOKINE protein or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with NEOKINE protein or target molecules but which do not interfere with binding of the NEOKINE protein to its target molecule can be derivatized to the wells of the plate, and unbound target or NEOKINE protein trapped in the wells by antibody conjugation. Methods for detecting such complexes, in addition to those described above for the GST-immobilized complexes, include immunodetection of complexes using antibodies reactive with the NEOKINE protein or target molecule, as well as enzyme-linked assays which rely on detecting an enzymatic activity associated with the NEOKINE protein or target molecule.

In another embodiment, modulators of NEOKINE expression are identified in a method wherein a cell is contacted with a candidate compound and the expression of NEOKINE mRNA or protein in the cell is determined. The level of expression of NEOKINE mRNA or protein in the presence of the candidate compound is compared to the level of expression of NEOKINE mRNA or protein in the absence of the candidate compound. The candidate compound can then be identified as a modulator of NEOKINE expression based on this comparison. For example, when expression of NEOKINE mRNA or protein is greater (statistically significantly greater) in the presence of the candidate compound than in its absence, the candidate compound is identified as a stimulator of NEOKINE mRNA or protein expression. Alternatively, when expression of NEOKINE mRNA or protein is less (statistically significantly less) in the presence of the candidate compound than in its absence, the candidate compound is identified as an inhibitor of NEOKINE mRNA or protein expression. The level of NEOKINE mRNA or protein expression in the cells can be determined by methods described herein for detecting NEOKINE mRNA or protein.

In yet another aspect of the invention, the NEOKINE proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with NEOKINE (“NEOKINE-binding proteins” or “NEOKINE-bp”) and are involved in NEOKINE activity. Such NEOKINE-binding proteins are also likely to be involved in the propagation of signals by the NEOKINE proteins as, for example, downstream elements of a NEOKINE-mediated signaling pathway. Alternatively, such NEOKINE-binding proteins are likely to be cell-surface molecules associated with non-NEOKINE expressing cells, wherein such NEOKINE-binding proteins are involved in chemoattraction.

The two-hybrid system is based on the modular nature of most transcription factors, which consist of separable DNA-binding and activation domains. Briefly, the assay utilizes two different DNA constructs. In one construct, the gene that codes for a NEOKINE protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NEOKINE-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the NEOKINE protein.

This invention further pertains to novel agents identified by the above-described screening assays. Accordingly, it is within the scope of this invention to further use an agent identified as described herein in an appropriate animal model. For example, an agent identified as described herein (e.g., a NEOKINE modulating agent, an antisense NEOKINE nucleic acid molecule, a NEOKINE-specific antibody, or a NEOKINE-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the NEOKINE nucleotide sequences, described herein, can be used to map the location of the NEOKINE genes on a chromosome. The mapping of the NEOKINE sequences to chromosomes is an important first step in correlating these sequences with genes associated with disease.

Briefly, NEOKINE genes can be mapped to chromosomes by preparing PCR primers (preferably 15-25 bp in length) from the NEOKINE nucleotide sequences. Computer analysis of the NEOKINE sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the NEOKINE sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NEOKINE nucleotide sequences to design oligonucleotide primers, sublocalization can be achieved with panels of fragments from specific chromosomes. Other mapping strategies which can similarly be used to map a 9o, 1p, or 1v sequence to its chromosome include in situ hybridization (described in Fan, Y. et al. (1990) PNAS, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et aL (1987) Nature, 325:783-787.

Moreover, differences in the DNA sequences between individuals affected and unaffected with a disease associated with the NEOKINE gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The NEOKINE sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the NEOKINE nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The NEOKINE nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1, can comfortably provide positive individual identification with a panel of perhaps 10 to 1,000 primers which each yield a noncoding amplified sequence of 100 bases. If predicted coding sequences, such as those in SEQ ID NO:3 are used, a more appropriate number of primers for positive individual identification would be 500-2,000.

If a panel of reagents from NEOKINE nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of Partial NEOKINE Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1 are particularly appropriate for this use as greater numbers of polymorphisms occur in the noncoding regions, making it easier to differentiate individuals using this technique. Examples of polynucleotide reagents include the NEOKINE nucleotide sequences or portions thereof, e.g., fragments derived from the noncoding regions of SEQ ID NO:1, having a length of at least 20 bases, preferably at least 30 bases.

The NEOKINE nucleotide sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such NEOKINE probes can be used to identify tissue by species and/or by organ type.

In a similar fashion, these reagents, e.g., NEOKINE primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining NEOKINE protein and/or nucleic acid expression as well as NEOKINE activity, in the context of a biological sample (e.g., blood, serum, cells, tissue) to thereby determine whether an individual is afflicted with a disease or disorder, or is at risk of developing a disorder, associated with aberrant NEOKINE expression or activity. The invention also provides for prognostic (or predictive) assays for determining whether an individual is at risk of developing a disorder associated with NEOKINE protein, nucleic acid expression or activity. For example, mutations in a NEOKINE gene can be assayed in a biological sample. Such assays can be used for prognostic or predictive purpose to thereby phophylactically treat an individual prior to the onset of a disorder characterized by or associated with NEOKINE protein, nucleic acid expression or activity.

Another aspect of the invention pertains to monitoring the influence of agents (e.g., drugs, compounds) on the expression or activity of NEOKINE in clinical trials.

These and other agents are described in further detail in the following sections.

1. Diagnostic Assays

An exemplary method for detecting the presence or absence of NEOKINE protein or nucleic acid in a biological sample involves obtaining a biological sample from a test subject and contacting the biological sample with a compound or an agent capable of detecting NEOKINE protein or nucleic acid (e.g., mRNA, genomic DNA) that encodes NEOKINE protein such that the presence of NEOKINE protein or nucleic acid is detected in the biological sample. A preferred agent for detecting NEOKINE mRNA or genomic DNA is a labeled nucleic acid probe capable of hybridizing to NEOKINE mRNA or genomic DNA. The nucleic acid probe can be, for example, a full-length NEOKINE nucleic acid, such as the nucleic acid of SEQ ID NO:1 (or that of SEQ ID NO:4, SEQ ID NO:7, SEQ ID NO:10, or the DNA insert of the plasmid deposited with ATCC as Accession Number 98751, or a portion thereof), such as an oligonucleotide of at least 15, 30, 50, 100, 250 or 500 nucleotides in length and sufficient to specifically hybridize under stringent conditions to NEOKINE mRNA or genomic DNA. Other suitable probes for use in the diagnostic assays of the invention are described herein.

A preferred agent for detecting NEOKINE protein is an antibody capable of binding to NEOKINE protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect NEOKINE mRNA, protein, or genomic DNA in a biological sample in vitro as well as in vivo. For example, in vitro techniques for detection of NEOKINE mRNA include Northern hybridizations and in situ hybridizations. In vitro techniques for detection of NEOKINE protein include enzyme linked immunosorbent assays (ELISAs), Western blots, immunoprecipitations and immunofluorescence. In vitro techniques for detection of NEOKINE genomic DNA include Southern hybridizations. Furthermore, in vivo techniques for detection of NEOKINE protein include introducing into a subject a labeled anti-NEOKINE antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting NEOKINE protein, mRNA, or genomic DNA, such that the presence of NEOKINE protein, mRNA or genomic DNA is detected in the biological sample, and comparing the presence of NEOKINE protein, mRNA or genomic DNA in the control sample with the presence of NEOKINE protein, mRNA or genomic DNA in the test sample.

The invention also encompasses kits for detecting the presence of NEOKINE in a biological sample. For example, the kit can comprise a labeled compound or agent capable of detecting NEOKINE protein or mRNA in a biological sample; means for determining the amount of NEOKINE in the sample; and means for comparing the amount of NEOKINE in the sample with a standard. The compound or agent can be packaged in a suitable container. The kit can further comprise instructions for using the kit to detect NEOKINE protein or nucleic acid.

2. Prognostic Assays

The diagnostic methods described herein can furthermore be utilized to identify subjects having or at risk of developing a disease or disorder associated with aberrant NEOKINE expression or activity. For example, the assays described herein, such as the preceding diagnostic assays or the following assays, can be utilized to identify a subject having or at risk of developing a disorder associated with NEOKINE protein, nucleic acid expression or activity such as an inflammatory disorder. Alternatively, the prognostic assays can be utilized to identify a subject having or at risk for developing an inflammatory disorder. Thus, the present invention provides a method for identifying a disease or disorder associated with aberrant NEOKINE expression or activity in which a test sample is obtained from a subject and NEOKINE protein or nucleic acid (e.g, mRNA, genomic DNA) is detected, wherein the presence of NEOKINE protein or nucleic acid is diagnostic for a subject having or at risk of developing a disease or disorder associated with aberrant NEOKINE expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NEOKINE expression or activity. For example, such methods can be used to determine whether a subject can be effectively treated with an agent for a disorder, such as an inflammatory disorder (e.g., kidney inflammation). Alternatively, such methods can be used to determine whether a subject can be effectively treated with an agent for an inflammatory disease. Thus, the present invention provides methods for determining whether a subject can be effectively treated with an agent for a disorder associated with aberrant NEOKINE expression or activity in which a test sample is obtained and NEOKINE protein or nucleic acid expression or activity is detected (e.g., wherein the abundance of NEOKINE protein or nucleic acid expression or activity is diagnostic for a subject that can be administered the agent to treat a disorder associated with aberrant NEOKINE expression or activity.)

The methods of the invention can also be used to detect genetic alterations in a NEOKINE gene, thereby determining if a subject with the altered gene is at risk for a disorder characterized by an aberrant inflammatory response. In preferred embodiments, the methods include detecting, in a sample of cells from the subject, the presence or absence of a genetic alteration characterized by at least one of an alteration affecting the integrity of a gene encoding a NEOKINE-protein, or the mis-expression of the NEOKINE gene. For example, such genetic alterations can be detected by ascertaining the existence of at least one of 1) a deletion of one or more nucleotides from a NEOKINE gene; 2) an addition of one or more nucleotides to a NEOKINE gene; 3) a substitution of one or more nucleotides of a NEOKINE gene, 4) a chromosomal rearrangement of a NEOKINE gene; 5) an alteration in the level of a messenger RNA transcript of a NEOKINE gene, 6) aberrant modification of a NEOKINE gene, such as of the methylation pattern of the genomic DNA, 7) the presence of a non-wild type splicing pattern of a messenger RNA transcript of a NEOKINE gene, 8) a non-wild type level of a NEOKINE-protein, 9) allelic loss of a NEOKINE gene, and 10) inappropriate post-translational modification of a NEOKINE-protein. As described herein, there are a large number of assay techniques known in the art which can be used for detecting alterations in a NEOKINE gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the NEOKINE-gene (see Abravaya et al. (1995) Nucleic Acids Res .23:675-682). This method can include the steps of collecting a sample of cells from a patient, isolating nucleic acid (e.g., genomic, mRNA or both) from the cells of the sample, contacting the nucleic acid sample with one or more primers which specifically hybridize to a NEOKINE gene under conditions such that hybridization and amplification of the NEOKINE-gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et all, 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NEOKINE gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in NEOKINE can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in NEOKINE can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential ovelapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NEOKINE gene and detect mutations by comparing the sequence of the sample NEOKINE with the corresponding wild-type (control) sequence. Examples of sequencing reactions include those based on techniques developed by Maxim and Gilbert ((1977) PNAS 74:560) or Sanger ((1977) PNAS 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the NEOKINE gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type NEOKINE sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NEOKINE cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a NEOKINE sequence, e.g., a wild-type NEOKINE sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in NEOKINE genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat Res 285:125-144; and Hayashi (1992) Genet Anal Tech Appl 9:73-79). Single-stranded DNA fragments of sample and control NEOKINE nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NEOKINE gene.

Furthermore, any cell type or tissue in which NEOKINE is expressed may be utilized in the prognostic assays described herein.

3. Monitoring of Effects During Clinical Trials

Monitoring the influence of agents (e.g., drugs, compounds) on the expression or activity of a NEOKINE protein (e.g., modulation of angiogenesis or of an inflammatory responese) an be applied not only in basic drug screening, but also in clinical trials. For example, the effectiveness of an agent determined by a screening assay as described herein to increase NEOKINE gene expression, protein levels, or upregulate NEOKINE activity, can be monitored in clinical trials of subjects exhibiting decreased NEOKINE gene expression, protein levels, or downregulated NEOKINE activity. Alternatively, the effectiveness of an agent determined by a screening assay to decrease NEOKINE gene expression, protein levels, or downregulate NEOKINE activity, can be monitored in clinical trials of subjects exhibiting increased NEOKINE gene expression, protein levels, or upregulated NEOKINE activity. In such clinical trials, the expression or activity of a NEOKINE gene, and preferably, other genes that have been implicated in, for example, an inflammatory disorder can be used as a “read out” or markers of the phenotype of a particular cell.

For example, and not by way of limitation, genes, including NEOKINE, that are modulated in cells by treatment with an agent (e.g., compound, drug or small molecule) which modulates NEOKINE activity (e.g., identified in a screening assay as described herein) can be identified. Thus, to study the effect of agents on inflammatory disorders, for example, in a clinical trial, cells can be isolated and RNA prepared and analyzed for the levels of expression of NEOKINE and other genes implicated in the inflammatory disorder, respectively. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NEOKINE or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of a NEOKINE protein, mRNA, or genomic DNA in the preadministration sample; (iii) obtaining one or more post-administration samples from the subject; (iv) detecting the level of expression or activity of the NEOKINE protein, mRNA, or genomic DNA in the post-administration samples; (v) comparing the level of expression or activity of the NEOKINE protein, mRNA, or genomic DNA in the pre-administration sample with the NEOKINE protein, mRNA, or genomic DNA in the post administration sample or samples; and (vi) altering the administration of the agent to the subject accordingly. For example, increased administration of the agent may be desirable to increase the expression or activity of NEOKINE to higher levels than detected, i.e., to increase the effectiveness of the agent. Alternatively, decreased administration of the agent may be desirable to decrease expression or activity of NEOKINE to lower levels than detected, i.e. to decrease the effectiveness of the agent. According to such an embodiment, NEOKINE expression or activity may be used as an indicator of the effectiveness of an agent, even in the absence of an observable phenotypic response.

C. Methods of Treatment:

The present invention provides for both prophylactic and therapeutic methods of treating a subject at risk of (or susceptible to) a disorder or having a disorder associated with aberrant NEOKINE expression or activity. Furthermore, the invention provides for both prophylactic and therapeutic methods of treating a subject at risk of (or susceptible to) a disorder or having a disorder associated with aberrant non-NEOKINE chemokine expression or activity. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the NEOKINE molecules of the present invention or NEOKINE modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant NEOKINE or chemokine expression or activity, by administering to the subject a NEOKINE or an agent which modulates NEOKINE expression or at least one NEOKINE or chemokine activity. Subjects at risk for a disease which is caused or contributed to by aberrant NEOKINE or chemokine expression or activity can be identified by, for example, any or a combination of diagnostic or prognostic assays as described herein. Administration of a prophylactic agent can occur prior to the manifestation of symptoms characteristic of the NEOKINE or chemokine aberrancy, such that a disease or disorder is prevented or, alternatively, delayed in its progression. Depending on the type of NEOKINE or chemokine aberrancy, for example, a NEOKINE, NEOKINE agonist or NEOKINE antagonist agent can be used for treating the subject. The appropriate agent can be determined based on screening assays described herein. The prophylactic methods of the present invention are further discussed in the following subsections.

2. Therapeutic Methods

Another aspect of the invention pertains to methods of modulating NEOKINE or chemokine expression or activity for therapeutic purposes. It has been determined that NEOKINE-1 is strongly expressed in the kidney. This expression of NEOKINE-1 indicates that the NEOKINES have utility in treating kidney inflammation, a major cause of renal failure in chronic and acute renal failure and transplantation. It is known in the art that expression of chemokines in the kidney is not only correlated with inflammation pathology, but also that blocking chemokine action by anti-chemokine antibodies limits or halts progression of the inflammation and resulting tissue damage. The fact that normal human kidney expresses abundant NEOKINE transcript suggests that the kidney makes significant quantities of the protein and that therefore the pro-inflammatory activity low or non-existent. This is consistent with the proposed natural antagonist function of the NEOKINE proteins. Furthermore, signal peptide cleavage prediction on NEOKINE implies that the mature protein will have only two residues before the first cysteine. In an analogous situation, an artificially truncated form of IL-8 with only one residue before the first cysteine instead of the naturally-occurring 6 residues converts the protein from an agonist to an antagonist.

Accordingly, in an exemplary embodiment, the modulatory method of the invention involves contacting a cell with a NEOKINE such that the activity of a chemokine is modulated. Alternatively, the modulatory method of the invention involves contacting a cell with a NEOKINE or agent that modulates one or more of the activities of NEOKINE protein activity associated with the cell. An agent that modulates NEOKINE protein activity can be an agent as described herein, such as a nucleic acid or a protein, a naturally-occurring target molecule of a NEOKINE protein (e.g., a carbohydrate), a NEOKINE antibody, a NEOKINE agonist or antagonist, a peptidomimetic of a NEOKINE agonist or antagonist, or other small molecule. In one embodiment, the agent stimulates one or more NEOKINE activites. Examples of such stimulatory agents include active NEOKINE protein and a nucleic acid molecule encoding NEOKINE that has been introduced into the cell. In another embodiment, the agent inhibits one or more NEOKINE activites. Examples of such inhibitory agents include antisense NEOKINE nucleic acid molecules and anti-NEOKINE antibodies. These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by aberrant expression or activity of a NEOKINE protein or nucleic acid molecule. Alternatively, the present invention provides methods of treating an individual afflicted with a disease or disorder characterized by aberrant expression or activity of a chemokine. In one embodiment, the method involves administering an agent (e.g., an agent identified by a screening assay described herein), or combination of agents that modulates (e.g., upregulates or downregulates) NEOKINE expression or activity or the expression or activity of a chemokine. In another embodiment, the method involves administering a NEOKINE protein or nucleic acid molecule as therapy to compensate for reduced or aberrant NEOKINE expression or activity. In another embodiment, the method involves administering a NEOKINE protein or nucleic acid molecule as therapy to compensate for reduced or aberrant chemokine expression or activity.

A preferred embodiment of the present invention involves a method for treatment of a NEOKINE associated disease or disorder which includes the step of administering a therapeutically effective amount of a NEOKINE antibody to a subject. As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment may increase or decrease over the course of a particular treatment. Changes in dosage may result from the results of diagnostic assays as described herein.

Stimulation of NEOKINE activity is desirable in situations in which NEOKINE is abnormally downregulated and/or in which increased NEOKINE activity is likely to have a beneficial effect. For example, stimulation of NEOKINE activity is desirable in situations in which a chemokine is upregulated and/or in which increased NEOKINE activity is likely to have a beneficial effect (e.g., a situation is where a subject has a disorder characterized by aberrant angiogenesis or inflammation, such as kidney inflammation. Likewise, inhibition of NEOKINE activity is desirable in situations in which NEOKINE is abnormally upregulated and/or in which decreased NEOKINE activity is likely to have a beneficial effect.

3. Pharmacogenomics

The NEOKINE molecules of the present invention, as well as agents, or modulators which have a stimulatory or inhibitory effect on NEOKINE activity (e.g., NEOKINE gene expression) as identified by a screening assay described herein can be administered to individuals to treat (prophylactically or therapeutically) disorders (e.g, inflammatory disorders) associated with aberrant NEOKINE activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a NEOKINE molecule or NEOKINE modulator as well as tailoring the dosage and/or therapeutic regimen of treatment with a NEOKINE molecule or NEOKINE modulator.

Pharmacogenomics deals with clinically significant hereditary variations in the response to drugs due to altered drug disposition and abnormal action in affected persons. See e.g., Eichelbaum, M., Clin Exp Pharmacol Physiol, 1996, 23(10-11):983-985 and Linder, M. W., Clin Chem, 1997, 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a NEOKINE protein or NEOKINE receptor of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., a NEOKINE molecule or NEOKIE modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a NEOKINE molecule or NEOKINE modulator, such as a modulator identified by one of the exemplary screening assays described herein.

This invention is further illustrated by the following examples which should not be construed as limiting. The contents of all references, patents and published patent applications cited throughout this application are hereby incorporated by reference.

### EXAMPLES

### Example 1

Identification And Characterization of NEOKINE-1 cDNAs

In this example, the identification and characterization of the genes encoding human, murine, rat and macaque NEOKINE-1 (also referred to as “ANTIKINE-1”, “TANGO 112”, or T112) is described.

Isolation of the Murine and Human NEOKINE-1 cDNAs

The invention is based, at least in part, on the discovery of the murine and human genes encoding a novel protein, referred to herein as NEOKINE-1. In order to identify potentially novel chemokines, using an automated procedure, the human amino acid sequences of the chemokines, interleukin-8, gamma-IP10, Sis-Delta, fractalkine, and SDF-1 were used to search proprietary databases and the dbEST databases using TBLASTN (Wash U. version, 2.0, BLOSUM62 search matrix). Sequences exhibiting 90% or greater identity to any protein present in Genpept, SwissProt, or PIR were marked as examples of these proteins and removed. This analysis identified a mouse EST (accession number AA013634) potentially encoding a chemokine. As the encoded protein was quite divergent from all other chemokine family members, and the open reading frame constituted a small percentage of the total cDNA length (see below), to establish whether this transcript encoded a novel chemokine family member, the nucleotide sequence of the entire cDNA was determined. To do this, first, additional ESTs from the mouse gene were retrieved from public databases by similarity searches. A total of 33 murine sequences were thus retrieved. Second, these sequences were used to create an extensive length of continuous sequence (contig). The 33 murine ESTs were aligned and edited at discrepant bases into a single contig of 1420 bp. The 5′ end of the contig appeared to be missing part of the open reading frame. To extend this sequence, the sizes of the inserts of several of the murine cDNA clones from which the ESTs were derived was determined and one with the largest insert (about 1.45 kb) was subsequently re-sequenced in its entirety. This extended the sequence by 5 bp to 1425 bp and corrected some remaining discrepancies.

72 human ESTs from a presumptive human orthologue of the above-described mouse gene were identified. However, these ESTs, after a similar assembly and editing, aligned into two non-overlapping contigs of 364 and 1101 bp. Comparison with the murine sequence suggested that the 364-bp contig derived from the 5′ part of the human gene, while the 1101-bp contig derived from the 3′ part of the human gene and included the poly(A) tail. The distribution of 5′ EST reads implied that there was no cDNA clone which contained both halves of the gene. At the 3′ end of the 5′ contig, there was a naturally occurring run of adenosine nucleotides (see below), to which the oligo-d(T) primer used in reverse transcription would have annealed (i.e., this primer would have annealed at two sites: the actual poly (A) tail and this internal oligo (A) stretch). This ectopic primer would have blocked reverse transcription in the human sequence at about 1.1 kb from the 3′ end, but also would have allowed a second set of cDNA molecules to be synthesized which covered the 5′ end of the gene. The two human EST contigs that can be derived from these 72 ESTs therefore would appear on inspection and in the absence of further information, to derive from two genes instead of one. That this is the case is shown by the fact that automated assembly of ESTs produced distinct “UniGene” numbers for the two contigs (Hs.21210 for the 5′ contig and Hs.24395 for the 3′ contig).

To establish that the two human contigs derived from the same transcript, two primers lacking oligo d(T) were designed from the two assembled human contigs such that they would amplify an ˜300 bp fragment spanning the contigs. Using first-strand cDNA prepared from human placental poly (A)+RNA, a unique ˜300 bp fragment aws amplified by standard techniques. This cDNA was subsequently cloned and sequenced. The sequence did span the two contigs and provided the missing sequence between them. The primers were h112/227f (CCAAGCGCTTCATCAAGTGG) (SEQ ID NO:11) and h112/526r (GCAGCCTGTGATGAAGTCTGG) (SEQ ID NO:12).

The 5′ end of the contig, which included part of the open reading frame, was missing from the assembled single contig. To obtain the complete transcript sequence at the 5′ end for the human gene, a cDNA clone (rthp112) extending from the 5′ end of the human transcript to beyond the end of the open reading frame was generated by the RACE procedure. The gene-specific primer used was t112racel (CAGCCTATTCTTCGTAGACCCTGC) (SEQ ID NO:13). The 5′-most 35 bp of this clone formed a palindrome and thus appeared to be a cloning artifact, as is well known in the art, and was removed from the final sequence. The remaining sequence extended the human cDNA sequence by 108 bp to 1564 bp and corrected some remaining discrepancies. Clone rthp112, comprising the entire coding region of human NEOKINE-1 has been deposited with the ATTC and has Accession No. 98751.

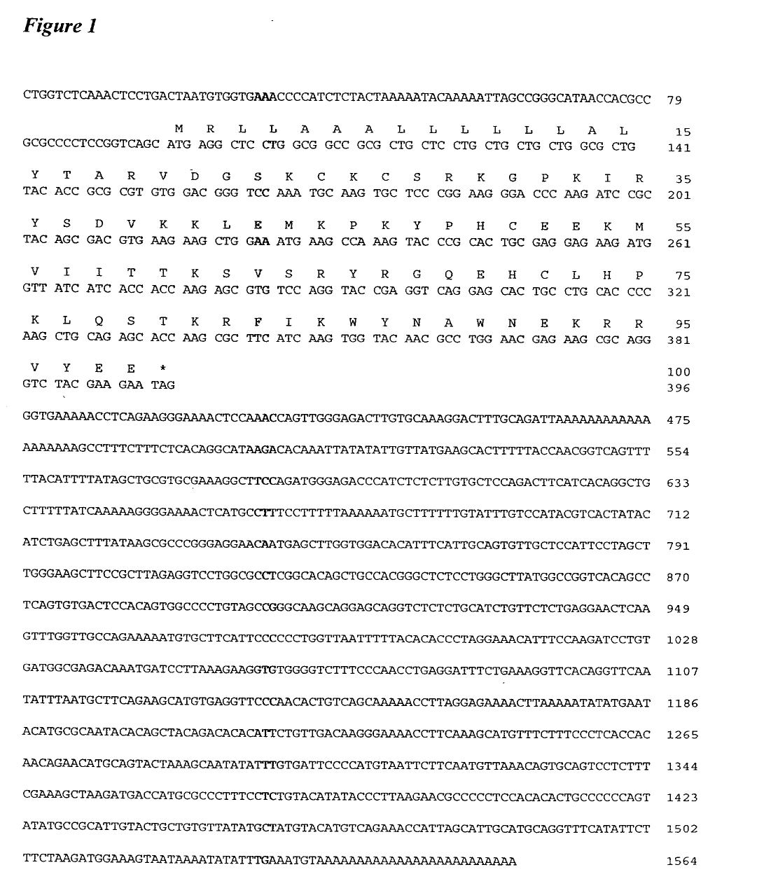

The nucleotide sequence encoding the human NEOKINE-1 protein is shown in FIG. 1 and is set forth as SEQ ID NO:1. The full length protein encoded by this nucleic acid is comprised of about 99 amino acids and has the amino acid sequence shown in FIG. 1 and set forth as SEQ ID NO:2. The coding portion (open reading frame) of SEQ ID NO:1 is set forth as SEQ ID NO:3.

To identify a murine cDNA clone containing a near full-length insert, the sizes of the inserts of several of the murine cDNA clones from which the ESTs were derived was determined and one with the largest insert (about 1.45 kb) was subsequently re-sequenced in its entirety. This extended the sequence by 5 bp to 1425 bp and corrected some remaining discrepancies.

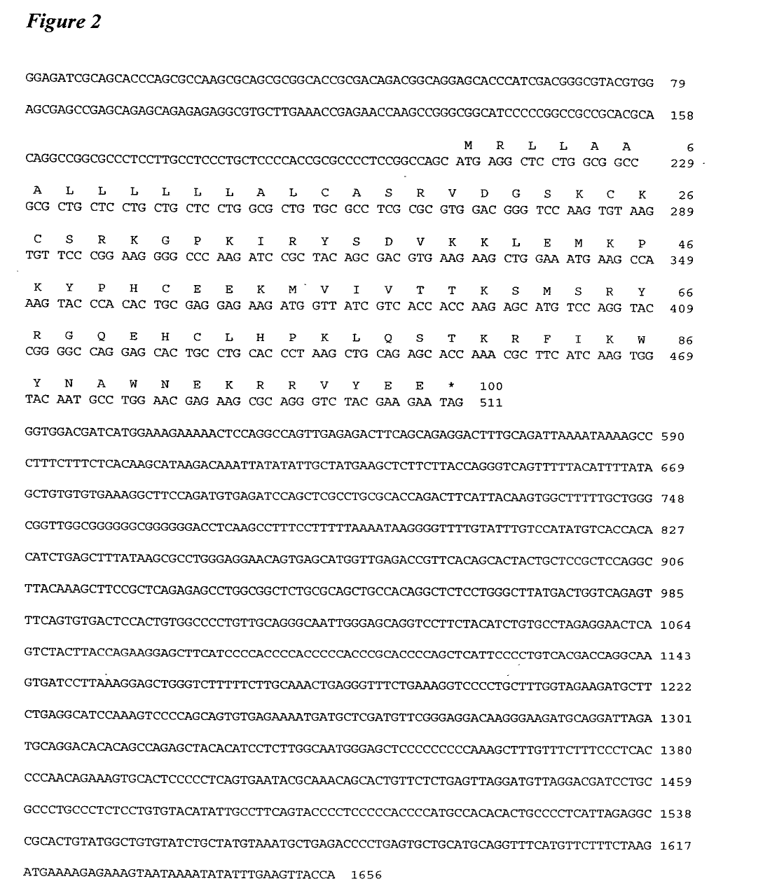

The nucleotide sequence encoding the murine NEOKINE-1 protein is shown in FIG. 2 and is set forth as SEQ ID NO: 4. The full length protein encoded by this nucleic acid is comprised of about 92 amino acids and has the amino acid sequence shown in FIG. 2 and set forth as SEQ ID NO:5. The coding portion (open reading frame) of SEQ ID NO:4 is set forth as SEQ ID NO:6.

Analysis of Murine and Human NEOKINE-1

Examination of the assembled and corrected cDNA sequences depicted in FIGS. 1 and 2 showed that they likely encoded highly-conserved proteins, human and mouse NEOKINE-1. Based on the presence of 4 cysteine residues, which presumably form 2 disulfide bonds, a predicted signal sequence, a predicted mature peptide mass of about 10,000 daltons, and a characteristic spacing of one residue between the first two cysteines, it was judged that the encoded protein was a novel member of the alpha chemokine family, and a member of the subfamily that lacked the glutamine-leucine-arginine sequence before the first cysteine. However, three atypical features were also present but conserved between species. These were, first, the presence of an extra 5 residues between the second pair of cysteines; second, the fewest residues before the predicted amino terminus of the mature protein and the first cysteine of any naturally-occurring chemokine; and third, a general dissimilarity to all other chemokines in the region between the second and third cysteines.

A BLAST search (Altschul et al. (1990) J. Mol. Biol. 215:403) of the nucleotide sequence of human NEOKINE-1 has revealed that NEOKINE-1 is significantly similar to a human STS (TIGR-A002I14, Accession No. G26440) which was sequenced as part of the WI/MIT human gene mapping project and derived from a TIGR-assembled contig that lacks any of the open reading frame of human NEOKINE-1. (The TIGR-assembled contig failed to reveal the true ORF of human NEOKINE-1, most likely to the existence of a significant number of potential ORFs which fortuitously exist in the long 3′ UTR of the human NEOKINE cDNA, but do not, in fact encode the human NEOKINE-1 protein.) The gene is located to human 5q31.1 near the marker D5S396, distinct from the chemokine cluster on chromosome 4q. Plausible human disease genes that map to this region include a hereditary eosinophilia (EOS) and a hereditary high serum IgE associated with hypersuppression of inflammation in the skin (IGES).

The clones of both the human and murine EST sequences used in the assembly of the human and murine contigs derive predominantly from prenatal tissues. In particlular, 6 human ESTs derive from clones isolated from neonatal female placenta, 4 human ESTs derive from clones isolated from 8-9-week placenta, 4 human ESTs derive from clones isolated from fetal heart, 4 human ESTs derive from clones isolated from 20-week male liver and spleen, 4 human ESTs derive from clones isolated from breast tumor, 4 human ESTs derive from clones isolated from colon tumor, 3 human ESTs derive from clones isolated from adult breast, 2 human ESTs derive from clones isolated from pregnant uterus, 2 human ESTs derive from clones isolated from endometrial tumor, 1 human EST derives from a clone isolated from fetal brain, 1 human EST derives from a clone isolated from alveolar rhabdomyosarcoma, 1 human EST derives from a clone isolated fromovary tumor, 1 human EST derives from a clone isolated from 8-9-week total fetus, 1 human EST derives from a clone isolated from TIGR placenta II, 1 human EST derives from a clone isolated from corneal stroma, 1 human EST derives from a clone isolated from 3 m muscular atrophy, 1 human EST derives from a clone isolated from thyroid tumor, 1 human EST derives from a clone isolated from a 6-week embryo, and 1 human EST derives from a clone isolated from a 12-week embryo.

11 murine ESTs derive from clones isolated from 13.5+14.5d whole embryo, 5 murine ESTs derive from clones isolated from 19.5d embryo, 4 murine ESTs derive from clones isolated from 8.5d embryo, 3 murine ESTs derive from clones isolated from 12.5d embryo, 3 murine ESTs derive from clones isolated from 7d kidney. 2 murine ESTs derive from clones isolated from 13.5+14.5d placenta, 1 murine EST derives from a clone isolated from 6.5/8.5d embryo, 1 murine EST derives from a clone isolated from liver, 1 murine EST derives from a clone isolated from 4-week male thymus, 1 murine EST derives from a clone isolated from diaphragm, and 1 murine EST derives from a clone isolated from 11-week skin.

Tissue Distribution of NEOKINE-1 mRNA

This Example describes the tissue distribution of NEOKINE mRNA, as determined by Northern blot and in situ hybridization.

Northern blot hybridizations with the various RNA samples were performed under standard conditions and washed under stringent conditions, i.e., 0.2×SSC at 65° C. In each sample, the probe hybridized to a single RNA of about 1.9 kb. The results of hybridization of the probe to various mRNA samples are described below.

Expression in mouse embryos was examined using a developmental mouse Northern (Clontech) and in situ hybridization. This blot had poly(A)+RNA isolated from whole embryos of age 7, 11, 15 and 17 days. The analysis using the mouse gene as a probe revealed intense expression of a unique 1.9 kb transcript in the 7 d sample, but this may be due to contaminating RNA from the placenta. In the remaining samples, expression was low at 11 d, highest on day 15, and then dropped again on day 17.

Expression in diverse human tissue was examined using tissue-specific Northern blots (Clontech). These blots had poly(A)+ RNA isolated from various disease-free human organs. The analysis using a PCR fragment of the human gene as a probe revealed strongest expression of a unique 1.9 kb transcript in the kidney and small intestine, followed by strong expression in the spleen, uterus and colon, and lower expression in thymus, prostate, stomach, thyroid, spinal cord, lymph node, trachea, adrenal gland, bone marrow, heart, brain placenta, liver, smooth muscle and pancreas. There was little/no expression in lung, peripheral blood leukocytes and testis. The size of the poly(A)+ transcript in both human and mouse (1.9 kb) is consistent with the length of the full-length cDNAs (1.56 kb) which lack the poly(A) tails.

In situ hybridization of a murine antisense probe to various embryonic, post-natal and adult tissues was performed as follows. 8 μm sagittal sections of fresh frozen embryonic day 14.5, 16.5 and postnatal day 1.5 B6 mice, as well as 8 μm sections of various adult B6 mouse tissues (listed below) were used for hybridization. Sections were postfixed with 4% formaldehyde in DEPC treated 1× phosphate-buffered saline at room temperature for 10 min before being rinsed twice in DEPC 1×phosphate-buffered saline and once in 0.1 M triethanolamine-HCl (pH 8.0). Following incubation in 0.25% acetic anhydride-0.1 M triethanolamine-HCl for 10 min, sections were rinsed in DEPC 2×SSC (1×SSC is 0.15M NaCl plus 0.015M sodium citrate). Tissue was dehydrated through a series of ethanol washes, incubated in 100% chloroform for 5 min, and then rinsed in 100% ethanol for 1 min and 95% ethanol for 1 min and allowed to air dry.

The hybridization was performed using a ³⁵S-radiolabeled cRNA (antisense) probe from the following DNA sequence,

| | |
|---|---|
| GTCCAAGTGTAAGTGTTCCCGGAAGGGGCCCAAGATCCGCTACAGCGACGT | (SEQ ID NO:14) |
| GAAGAAGCTGGAAATGAAGCCAAAGTACCCACACTGCGAGGAGAAGATGG | |
| TTATCGTCACCACCAAGAGCATGTCCAGGTACCGGGGCCAGGAGCACTGCC | |
| TGCACCCTAAGCTGCAGAGCACCAAACGCTTCATCAAGTGGTACAATGCCTG | |
| GAACGAGAAGCGCAGGGTCTACGAAGAATAGGGTGGACGATCATGGAAAG | |
| AAAAACTCCAGGCCAGTTGAGAGACTTCAGCAGAGGACTTTGCAGATTAAA | |
| ATAAAAGCCCTTTCTTTCTCACAAGCATAAGACAAATTATATATTGCTATGA | |
| AGCTCTTCTTACCAGGGTCAGTTTTTACATTTTATAGCTGTGTGTGAAAGGCT | |
| TCCAGATGTGAGATCCAGCTCGCCTGCGCACCAGACTTCATTACAAGTGGCT | |
| TTTTGCTGGGCGGTTG. | |

A sense RNA probe made from the same DNA sequence was used to determine specificity of the antisense probe. Tissues were incubated with probes (approximately 5×10⁷ cpm/ml) in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type ×1, 1×Denhardt's solution, 50% formamide, 10% dextran sulfate, 100 mM dithiothreitol, 0.1% sodium dodecyl sulfate (SDS), and 0.1% sodium thiosulfate for 18 h at 55° C. After hybridization, slides were washed with 2×SSC. Sections were then sequentially incubated at 37° C. in TNE (a solution containing 10 MM Tris-HCl (pH 7.6), 500 mM NaCl, and 1 mM EDTA), for 10 min, in TNE with 10 μg of RNase A per ml for 30 min, and finally in TNE for 10 min. Slides were then rinsed with 2×SSC at room temp, washed with 2×SSC at 50° C. for 1 h, washed with 0.2×SSC at 55° C. for 1 h, and 0.2×SSC at 60° C. for 1 h. Sections were then dehydrated rapidly through serial ethanol-0.3 M sodium acetate concentrations before being air dried and exposed to Kodak Biomax MR scientific imaging film for 4 days at room temperature.

Following a 4 day film exposure, NEOKINE mRNA was detectable in the following tissues (Note: Tissues incubated with sense probe showed no signal in any tissues):

Adult Mouse:

Brain—multifocal signal in the cortex Purkinje cell layer of the cerebellum areas of the hippocampus and forebrain a small discrete region in the ventral portion of the hindbrain low level ubiquitous signal in most other regions

eye—multifocal signal seen in harderian gland anterior surface of lens/cornea retina

descending colon—focal signal

transverse/ascending colon—multifocal signal

small Intestine—villi

kidney—cortical region

adrenal gland—medulla region and capsule

heart—multifocal signal

skeletal muscle—multifocal signal

lung—signal outlining the large airways

thymus—low signal

bladder—high signal from the transitional epithelium

placenta—signal seen in the outer membrane/cell layer

The following tissues were tested but no signal was detected in:

spleen

liver

Postnatal Day 1.5 Mouse:

brain—specific regions, most notably the cortex no ubiquitous expression as seen in adult

nasal turbinates

developing upper and lower teeth

trachea

uterus

sternebral cartilage

kidney—medulla and outermost cortex in a multifocal pattern

skin and hair follicles—very strong signal

intestine

note: no signal observed in lung

Embryonic Day 16.5 Mouse:

brain—specific regions, most notably the cortex no ubiquitous expression as seen in adult

spinal cord—low signal

esophagus

lung—signal from large airways

adrenal gland—cortical region (note: opposite of adult)

kidney—medulla and outermost cortex in a multifocal pattern

skin—very strong signal

intestine

Embryonic Day 14.5 Mouse:

brain—discrete regions, most notable a region in the hindbrain

lung—signal from large airways

skin—very strong signal especially from whisker pads and tip of nose and tail

umbilical cord

intestine

These results reveal a striking distribution of NEOKINE mRNA in various non-lymphoid organs. The fact that many normal tissues express abundant NEOKINE transcript suggests that these tissues make significant quantities of NEOKINE protein and that therefore the pro-inflammatory or chemoattractive activities of NEOKINE are very low or non-existent. Indeed, NEOKINE is expressed most significantly in highly immunoreactive tissues that are involved in barrier functions. This observation is consistent with a proposed role of NEOKINE as a suppressor of inflammation. Furthermore, the predicted amino terminus of mature NEOKINE lies just two residues from the first cysteine. In an analogous situation, an artificially truncated form of human IL-8 with only one residue before the first cysteine instead of the normal 6 residues converts the protein from an agonist to an antagonist of its cognate receptor. These observations are further consistent with the proposed anti-inflammatory activity being mediated by antagonizing the action of other pro-inflammatory chemokines. The general divergence of NEOKINE from other alpha chemokines while being highly conserved itself, and the presence of the extra 5 residues between the second pair of cysteines could also be consistent with a broad antagonist function on a diverse set of chemokine receptors. The strong expression in the skin, the kidney, the bronchial and the brain indicates that NEOKINES have utility in treating inflammation of these organs, such as occurs in acute renal failure, transplantation, allergy and infection. Furthermore, since the human AIDS virus HIV uses some chemokine receptors as co-receptors for infection, NEOKINES may also have utility in slowing or blocking infection by HIV.

### Example 2

Isolation and Characterization of Rat and Macaque NEOKINE-1 cDNAs

During routine tests for sequences similar to mouse or human NEOKINE cDNA, one EST deriving from a rat brain cDNA and three ESTs deriving from macaque brain cDNAs were identified in a database of proprietary sequences. The database derives, at least in part, from sequencing of various mammalian clones generated from cDNA libraries created according to routine procedures. The cDNAs originated from rat brain and macaque brain libraries, respectively.

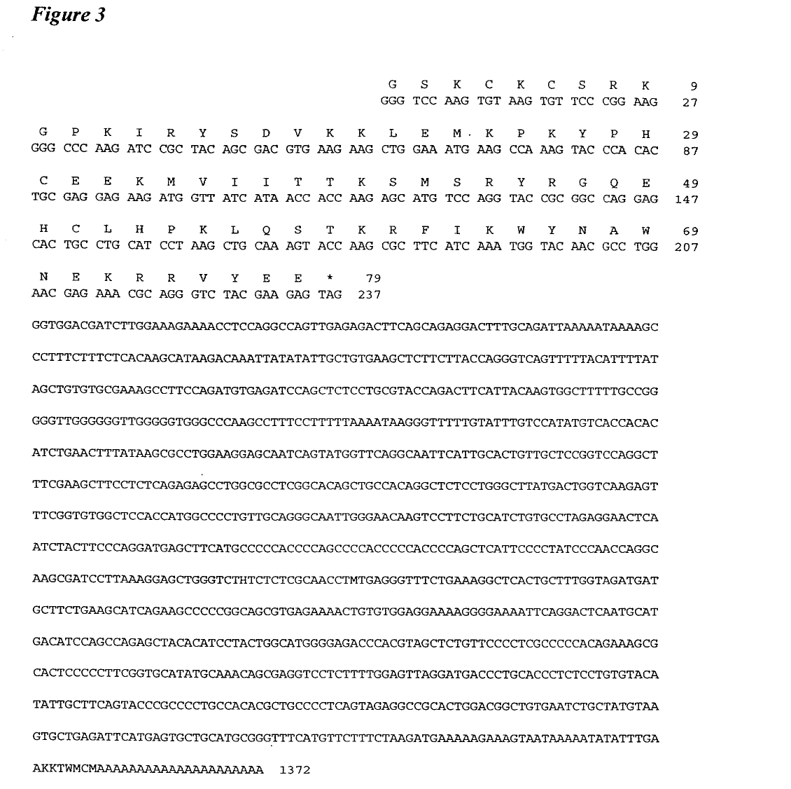

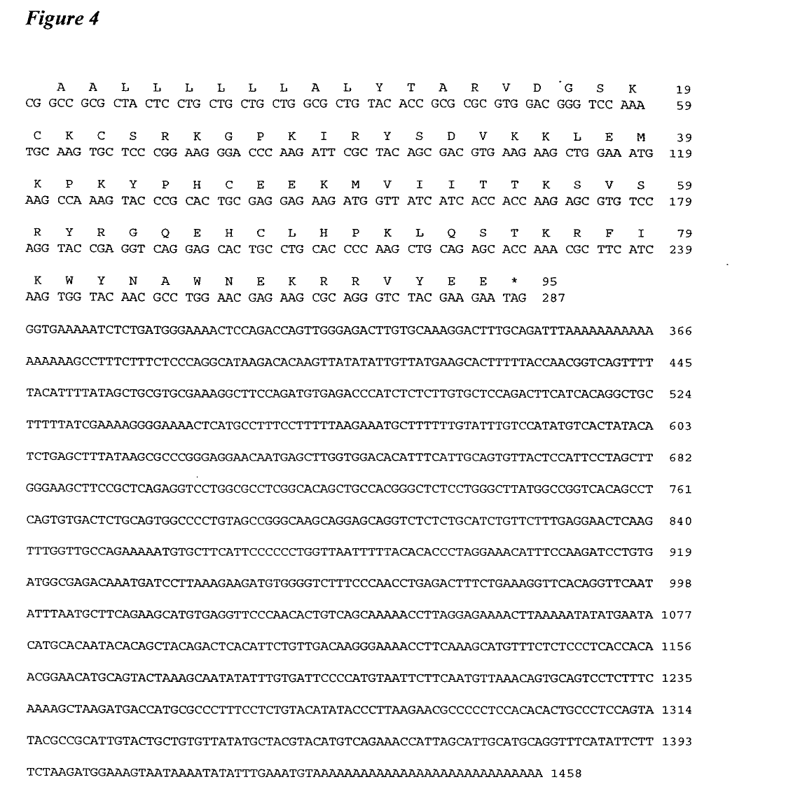

The nucleotide sequence encoding the at least 78 amino acid residues of the rat NEOKINE-1 protein (corresponding to the predicted mature protein) is shown in FIG. 3 and is set forth as SEQ ID NO: 7. The amino acid sequence for mature rat NEOKINE-1 is shown in FIG. 3 and set forth as SEQ ID NO:8. The coding portion (open reading frame) of SEQ ID NO:7 is set forth as SEQ ID NO:9. The nucleotide sequence and predicted amino acid sequence of macaque NEOKINE-1 are shown in FIG. 4 and set forth as SEQ ID NO:10 and SEQ ID NO:21, respectively.

Examination of the cDNA sequence depicted in FIGS. 3 shows that rat NEOKINE-1 comprises four cysteine residues which are conserved among all NEOKINE-1 family members identified thus far. Structural analysis of these proteins indicates that these cysteines are capable of forming 2 disulfide bridges. FIG. 5 depicts the alignment between the four NEOKINE-1 amino acid sequences identified according to these Examples.

### Example 3

Secretion of NEOKINE Chemokines

Expression constructs for RGSHis6 epitope-tagged (C-terminus) human NEOKINE were transfected into 293T cells using lipofectamine (GIBCO/BRL) according to the manufacturers instructions. After culturing in appropriate medium for 48-72 hours, conditioned medium was harvested, spun, filtered, and passed over nickel metal chelating column (Qiagen). After washing, bound material was eluted 200 mM imidazole buffer and fractions collected. Peak fractions were analyzed by SDS-PAGE and western blot using anti His6 antibodies (Quiagen). Purified NEOKINE protein bound to PVDF membrane after SDS-PAGE and electroblotting was sequenced for N-terminal amino acid analysis using Edman-based chemistry protein sequencing. The amino acid residues were analyzed by HPLC and determined by separation and peak height as compared to standards.

The N-terminal sequence of band NEOKINE was found to be SKCKCSRKGP which corresponds exactly to the predicted signal peptide cleavage site (between Gly22 and Ser23). Because the same band is identified by anti-His6 antibodies, which recognize the C-terminal epitope tag, the band was identified as the full length, mature NEOKINE protein.

### Example 4

Binding of NEOKINE to the NEOKINE Receptor (e.g., RDC1)

T112 (NEOKINE) was radioiodinated with lactoperoxidase according to standard protocols. RDC-1 in vector Pcdna3.1 was transiently transfected into 293 cells using calcium phosphate precipitation methodology. 72 hours after transfection, cells were harvested and binding assays performed under standard binding conditions for chemokines (e.g., low salt binding, high salt wash, binding at 24° C., 1 hour). Cells were then pelleted, washed, and radioactivity was counted. Binding was demonstrated and was determined to be high affinity by competition with unlabelled T112. The cpm bound in cell pellets in indicated below.

| | | | |
|---|---|---|---|
| ¹²⁵I-T112 | 0.1 nm | 37613.0 cpm | 27014.0 cpm |
| T112 | 3.0 nm | 8343.0 cpm | 9229.0 cpm |

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. An isolated nucleic acid molecule selected from the group consisting of:
a) a nucleic acid molecule comprising the nucleotide sequence of SEQ ID NO:4, SEQ ID NO:6, SEQ ID NO:7, SEQ ID NO:9, SEQ ID NO:10 or SEQ ID NO:22, or a complement thereof;
b) a nucleic acid molecule comprising a fragment of at least 100 contiguous nucleotides of a nucleic acid comprising the nucleotide sequence of SEQ ID NO:4, SEQ ID NO:6, SEQ ID NO:7, SEQ ID NO:9, SEQ ID NO:10, or SEQ ID NO:22, or a complement thereof;
c) a nucleic acid molecule which encodes a polypeptide comprising the amino acid sequence of SEQ ID NO:5, SEQ ID NO:8 or SEQ ID NO:21;
d) a nucleic acid molecule which encodes a fragment of a polypeptide comprising the amino acid sequence of SEQ ID NO:5, SEQ ID NO:8 or SEQ ID NO:21, wherein the fragment comprises at least 15 contiguous amino acid residues of the amino acid sequence of SEQ ID NO:5, SEQ ID NO:8 or SEQ ID NO:21; and
e) a nucleic acid molecule which encodes a naturally occurring allelic variant of a polypeptide comprising the amino acid sequence of SEQ ID NO:5, SEQ ID NO:8 or SEQ ID NO:21, wherein the nucleic acid molecule hybridizes to a nucleic acid molecule comprising SEQ ID NO:4, SEQ ID NO:6, SEQ ID NO:7, SEQ ID NO:9, SEQ ID NO:10, or SEQ ID NO:22 under stringent conditions.

2. The nucleic acid molecule of claim 1 further comprising vector nucleic acid sequences.

3. The nucleic acid molecule of claim 1 further comprising nucleic acid sequences encoding a heterologous polypeptide.

4. A host cell which contains the nucleic acid molecule of claim 1.

5. The host cell of claim 4 which is a mammalian host cell.

6. A non-human mammalian host cell containing the nucleic acid molecule of claim 1.

7. An isolated polypeptide selected from the group consisting of:
a) a fragment of a polypeptide comprising the amino acid sequence of SEQ ID NO:5, SEQ ID NO:8 or SEQ ID NO:21, wherein the fragment comprises at least 15 contiguous amino acids of SEQ ID NO:5, SEQ ID NO:8 or SEQ ID NO:21;
b) a naturally occurring allelic variant of a polypeptide comprising the amino acid sequence of SEQ ID NO:5, SEQ ID NO:8 or SEQ ID NO:21, wherein the polypeptide is encoded by a nucleic acid molecule which hybridizes to a nucleic acid molecule comprising SEQ ID NO:4, SEQ ID NO:6, SEQ ID NO:7, SEQ ID NO:9, SEQ ID NO:10, or SEQ ID NO:22 under stringent conditions;
c) a polypeptide which is encoded by the nucleic acid molecule comprising the nucleotide sequence of SEQ ID NO:4, SEQ ID NO:6, SEQ ID NO:7, SEQ ID NO:9, SEQ ID NO:10, or SEQ ID NO:22; and
d) a polypeptide comprising the amino acid sequence of SEQ ID NO:5, SEQ ID NO:8 or SEQ ID NO:21.

8. The polypeptide of claim 7 further comprising heterologous amino acid sequences.

9. An antibody which selectively binds to a polypeptide of claim 7.

10. A method for producing a polypeptide selected from the group consisting of:
a) a polypeptide comprising the amino acid sequence of SEQ ID NO:5, SEQ ID NO:8 or SEQ ID NO:21;
b) a fragment of a polypeptide comprising the amino acid sequence of SEQ ID NO:5, SEQ ID NO:8 or SEQ ID NO:21, wherein the fragment comprises at least 15 contiguous amino acids of SEQ ID NO:5, SEQ ID NO:8 or SEQ ID NO:21; and
c) a naturally occurring allelic variant of a polypeptide comprising the amino acid sequence of SEQ ID NO:5, SEQ ID NO:8 or SEQ ID NO:21, wherein the polypeptide is encoded by a nucleic acid molecule which hybridizes to a nucleic acid molecule comprising SEQ ID NO:4, SEQ ID NO:6, SEQ ID NO:7, SEQ ID NO:9, SEQ ID NO:10, or SEQ ID NO:22 under stringent conditions;
comprising culturing the host cell of claim 4 under conditions in which the nucleic acid molecule is expressed.

11. A method for detecting the presence of a polypeptide of claim 7 in a sample comprising:
a) contacting the sample with a compound which selectively binds to the polypeptide; and
b) determining whether the compound binds to the polypeptide in the sample to thereby detect the presence of a polypeptide of claim 7 in the sample.

12. The method of claim 11, wherein the compound which binds to the polypeptide is an antibody.

13. A kit comprising a compound which selectively binds to a polypeptide of claim 7 and instructions for use.

14. A method for detecting the presence of a nucleic acid molecule in claim 1 in a sample comprising:
a) contacting the sample with a nucleic acid probe or primer which selectively hybridizes to the nucleic acid molecule; and
b) determining whether the nucleic acid probe or primer binds to a nucleic acid molecule in the sample to thereby detect the presence of a nucleic acid molecule of claim 1 in the sample.

15. The method of claim 14, wherein the sample comprises mRNA molecules and is contacted with a nucleic acid probe.

16. A kit comprising a compound which selectively hybridizes to a nucleic acid molecule of claim 1 and instructions for use.

17. A method for identifying a compound which modulates binding of NEOKINE to NEOKINE receptor comprising:
a) contacting a cell which expresses NEOKINE receptor with NEOKINE and a test compound under conditions which allow NEOKINE to bind to NEOKINE receptor; and
b) detecting binding of NEOKINE in the presence of the test compound;
wherein a change in the amount of NEOKINE binding to NEOKINE receptor detected in the presence of the test compound indicates that the test compound modulates binding of NEOKINE to NEOKINE receptor.

18. A method for identifying a compound which modulates the activity of NEOKINE receptor comprising:
a) contacting a cell which expresses NEOKINE receptor with NEOKINE and a test compound under conditions which allow NEOKINE to bind to NEOKINE receptor; and
b) detecting the activity of NEOKINE receptor in the presence of the test compound;
wherein a change in the activity NEOKINE receptor detected in the presence of the test compound indicates that the test compound modulates the activity of NEOKINE receptor.

19. A method for identifying a compound which modulates binding of NEOKINE to NEOKINE receptor comprising:
a) contacting NEOKINE receptor with NEOKINE and a test compound under conditions which allow NEOKINE to bind to NEOKINE receptor; and
b) detecting binding of NEOKINE in the presence of the test compound;
wherein a change in the amount of NEOKINE binding to NEOKINE receptor detected in the presence of the test compound indicates that the test compound modulates binding of NEOKINE to NEOKINE receptor.

20. The method of any of claims 17 to 19 wherein the NEOKINE receptor is selected from the group consisting of:
a) a receptor having an amino acid sequence which is substantially homologous to the amino acid sequence of SEQ ID NO:16, SEQ ID NO:18, or SEQ ID NO:20;
b) a receptor which is encoded by an isolated nucleic acid molecule which is substantially homologous to the nucleic acid sequence of SEQ ID NO:15, SEQ ID NO:17, or SEQ ID NO:19; and
c) a receptor which is encoded by an isolated nucleic acid molecule which hybridizes under stringent conditions to a nucleic acid molecule having the nucleic acid sequence of SEQ ID NO:15, SEQ ID NO:17, or SEQ ID NO:17.

21. The method of any of claims 17 to 19 wherein the NEOKINE is selected form the group consisting of:
a) a polypeptide having an amino acid sequence which is substantially homologous to the amino acid sequence of SEQ ID NO:2, SEQ ID NO:5, or SEQ ID NO:8;
b) a polypeptide which is encoded by an isolated nucleic acid molecule which is substantially homologous to the nucleic acid sequence of SEQ ID NO:15, SEQ ID NO:17, or SEQ I) NO:19; and
c) a polypeptide which is encoded by an isolated nucleic acid molecule which hybridizes under stringent conditions to a nucleic acid molecule having the nucleic acid sequence of SEQ ID NO:15, SEQ ID NO:17, or SEQ ID NO:17.

22. A method of modulating the activity of a polypeptide of claim 7 comprising contacting the polypeptide or a cell expressing the polypeptide with a compound which binds to the polypeptide in a sufficient concentration to modulate the activity of the polypeptide.


SEQUENCE LISTING (22 sequences)

SEQ ID NO: 1 - DNA, 1564 bp, Homo sapiens
  CDS (97)..(393)
ctggtctcaaactcctgactaatgtggtgaaaccccatctctactaaaaatacaaaaattagccgggcataaccacgccgcgcccctccggtcagcatgaggctcctggcggccgcgctgctcctgctgctgctggcgctgtacaccgcgcgtgtggacgggtccaaatgcaagtgctcccggaagggacccaagatccgctacagcgacgtgaagaagctggaaatgaagccaaagtacccgcactgcgaggagaagatggttatcatcaccaccaagagcgtgtccaggtaccgaggtcaggagcactgcctgcaccccaagctgcagagcaccaagcgcttcatcaagtggtacaacgcctggaacgagaagcgcagggtctacgaagaatagggtgaaaaacctcagaagggaaaactccaaaccagttgggagacttgtgcaaaggactttgcagattaaaaaaaaaaaaaaaaaaagcctttctttctcacaggcataagacacaaattatatattgttatgaagcactttttaccaacggtcagtttttacattttatagctgcgtgcgaaaggcttccagatgggagacccatctctcttgtgctccagacttcatcacaggctgctttttatcaaaaaggggaaaactcatgcctttcctttttaaaaaatgcttttttgtatttgtccatacgtcactatacatctgagctttataagcgcccgggaggaacaatgagcttggtggacacatttcattgcagtgttgctccattcctagcttgggaagcttccgcttagaggtcctggcgcctcggcacagctgccacgggctctcctgggcttatggccggtcacagcctcagtgtgactccacagtggcccctgtagccgggcaagcaggagcaggtctctctgcatctgttctctgaggaactcaagtttggttgccagaaaaatgtgcttcattcccccctggttaatttttacacaccctaggaaacatttccaagatcctgtgatggcgagacaaatgatccttaaagaaggtgtggggtctttcccaacctgaggatttctgaaaggttcacaggttcaatatttaatgcttcagaagcatgtgaggttcccaacactgtcagcaaaaaccttaggagaaaacttaaaaatatatgaatacatgcgcaatacacagctacagacacacattctgttgacaagggaaaaccttcaaagcatgtttctttccctcaccacaacagaacatgcagtactaaagcaatatatttgtgattccccatgtaattcttcaatgttaaacagtgcagtcctctttcgaaagctaagatgaccatgcgccctttcctctgtacatatacccttaagaacgccccctccacacactgccccccagtatatgccgcattgtactgctgtgttatatgctatgtacatgtcagaaaccattagcattgcatgcaggtttcatattctttctaagatggaaagtaataaaatatatttgaaatgtaaaaaaaaaaaaaaaaaaaaaaaaa

SEQ ID NO: 2 - protein, 99 aa, Homo sapiens
MRLLAAALLLLLLALYTARVDGSKCKCSRKGPKIRYSDVKKLEMKPKYPHCEEKMVIITTKSVSRYRGQEHCLHPKLQSTKRFIKWYNAWNEKRRVYEE

SEQ ID NO: 3 - DNA, 300 bp, Homo sapiens
  CDS (1)..(297)
atgaggctcctggcggccgcgctgctcctgctgctgctggcgctgtacaccgcgcgtgtggacgggtccaaatgcaagtgctcccggaagggacccaagatccgctacagcgacgtgaagaagctggaaatgaagccaaagtacccgcactgcgaggagaagatggttatcatcaccaccaagagcgtgtccaggtaccgaggtcaggagcactgcctgcaccccaagctgcagagcaccaagcgcttcatcaagtggtacaacgcctggaacgagaagcgcagggtctacgaagaatag

SEQ ID NO: 4 - DNA, 1656 bp, Mus musculus
  CDS (212)..(508)
ggagatcgcagcacccagcgccaagcgcagcgcggcaccgcgacagacggcaggagcacccatcgacgggcgtacgtggagcgagccgagcagagcagagagaggcgtgcttgaaaccgagaaccaagccgggcggcatcccccggccgccgcacgcacaggccggcgccctccttgcctccctgctccccaccgcgcccctccggccagcatgaggctcctggcggccgcgctgctcctgctgctcctggcgctgtgcgcctcgcgcgtggacgggtccaagtgtaagtgttcccggaaggggcccaagatccgctacagcgacgtgaagaagctggaaatgaagccaaagtacccacactgcgaggagaagatggttatcgtcaccaccaagagcatgtccaggtaccggggccaggagcactgcctgcaccctaagctgcagagcaccaaacgcttcatcaagtggtacaatgcctggaacgagaagcgcagggtctacgaagaatagggtggacgatcatggaaagaaaaactccaggccagttgagagacttcagcagaggactttgcagattaaaataaaagccctttctttctcacaagcataagacaaattatatattgctatgaagctcttcttaccagggtcagtttttacattttatagctgtgtgtgaaaggcttccagatgtgagatccagctcgcctgcgcaccagacttcattacaagtggctttttgctgggcggttggcggggggcggggggacctcaagcctttcctttttaaaataaggggttttgtatttgtccatatgtcaccacacatctgagctttataagcgcctgggaggaacagtgagcatggttgagaccgttcacagcactactgctccgctccaggcttacaaagcttccgctcagagagcctggcggctctgcgcagctgccacaggctctcctgggcttatgactggtcagagtttcagtgtgactccactgtggcccctgttgcagggcaattgggagcaggtccttctacatctgtgcctagaggaactcagtctacttaccagaaggagcttcatccccaccccacccccacccgcaccccagctcattcccctgtcacgaccaggcaagtgatccttaaaggagctgggtctttttcttgcaaactgagggtttctgaaaggtcccctgctttggtagaagatgcttctgaggcatccaaagtccccagcagtgtgagaaaatgatgctcgatgttcgggaggacaagggaagatgcaggattagatgcaggacacacagccagagctacacatcctcttggcaatgggagctcccccccccaaagctttgtttctttccctcaccccaacagaaagtgcactccccctcagtgaatacgcaaacagcactgttctctgagttaggatgttaggacgatcctgcgccctgccctctcctgtgtacatattgccttcagtacccctcccccaccccatgccacacactgcccctcattagaggccgcactgtatggctgtgtatctgctatgtaaatgctgagacccctgagtgctgcatgcaggtttcatgttctttctaagatgaaaagagaaagtaataaaatatatttgaagttacca

SEQ ID NO: 5 - protein, 99 aa, Mus musculus
MRLLAAALLLLLLALCASRVDGSKCKCSRKGPKIRYSDVKKLEMKPKYPHCEEKMVIVTTKSMSRYRGQEHCLHPKLQSTKRFIKWYNAWNEKRRVYEE

SEQ ID NO: 6 - DNA, 300 bp, Mus musculus
  CDS (1)..(297)
atgaggctcctggcggccgcgctgctcctgctgctcctggcgctgtgcgcctcgcgcgtggacgggtccaagtgtaagtgttcccggaaggggcccaagatccgctacagcgacgtgaagaagctggaaatgaagccaaagtacccacactgcgaggagaagatggttatcgtcaccaccaagagcatgtccaggtaccggggccaggagcactgcctgcaccctaagctgcagagcaccaaacgcttcatcaagtggtacaatgcctggaacgagaagcgcagggtctacgaagaatag

SEQ ID NO: 7 - DNA, 1372 bp, Rattus norvegicus
  CDS (1)..(234)
gggtccaagtgtaagtgttcccggaaggggcccaagatccgctacagcgacgtgaagaagctggaaatgaagccaaagtacccacactgcgaggagaagatggttatcataaccaccaagagcatgtccaggtaccgcggccaggagcactgcctgcatcctaagctgcaaagtaccaagcgcttcatcaaatggtacaacgcctggaacgagaaacgcagggtctacgaagagtagggtggacgatcttggaaagaaaacctccaggccagttgagagacttcagcagaggactttgcagattaaaaataaaagccctttctttctcacaagcataagacaaattatatattgctgtgaagctcttcttaccagggtcagtttttacattttatagctgtgtgcgaaagccttccagatgtgagatccagctctcctgcgtaccagacttcattacaagtggctttttgccgggggttggggggttgggggtgggcccaagcctttcctttttaaaataagggtttttgtatttgtccatatgtcaccacacatctgaactttataagcgcctggaaggagcaatcagtatggttcaggcaattcattgcactgttgctccggtccaggctttcgaagcttcctctcagagagcctggcgcctcggcacagctgccacaggctctcctgggcttatgactggtcaagagtttcggtgtggctccaccatggcccctgttgcagggcaattgggaacaagtccttctgcatctgtgcctagaggaactcaatctacttcccaggatgagcttcatgcccccaccccagccccacccccaccccagctcattcccctatcccaaccaggcaagcgatccttaaaggagctgggtcthtctctcgcaacctmtgagggtttctgaaaggctcactgctttggtagatgatgcttctgaagcatcagaagcccccggcagcgtgagaaaactgtgtggaggaaaaggggaaaattcaggactcaatgcatgacatccagccagagctacacatcctactggcatggggagacccacgtagctctgttcccctcgcccccacagaaagcgcactcccccttcggtgcatatgcaaacagcgaggtcctcttttggagttaggatgaccctgcaccctctcctgtgtacatattgcttcagtacccgcccctgccacacgctgcccctcagtagaggccgcactggacggctgtgaatctgctatgtaagtgctgagattcatgagtgctgcatgcgggtttcatgttctttctaagatgaaaaagaaagtaataaaaatatatttgaakktwmcmaaaaaaaaaaaaaaaaaaaaa

SEQ ID NO: 8 - protein, 78 aa, Rattus norvegicus
GSKCKCSRKGPKIRYSDVKKLEMKPKYPHCEEKMVIITTKSMSRYRGQEHCLHPKLQSTKRFIKWYNAWNEKRRVYEE

SEQ ID NO: 9 - DNA, 237 bp, Rattus norvegicus
  CDS (1)..(234)
gggtccaagtgtaagtgttcccggaaggggcccaagatccgctacagcgacgtgaagaagctggaaatgaagccaaagtacccacactgcgaggagaagatggttatcataaccaccaagagcatgtccaggtaccgcggccaggagcactgcctgcatcctaagctgcaaagtaccaagcgcttcatcaaatggtacaacgcctggaacgagaaacgcagggtctacgaagagtag

SEQ ID NO: 10 - DNA, 1458 bp, Unknown
  CDS (3)..(284)
  Description of Unknown Organismmacaque
cggccgcgctactcctgctgctgctggcgctgtacaccgcgcgcgtggacgggtccaaatgcaagtgctcccggaagggacccaagattcgctacagcgacgtgaagaagctggaaatgaagccaaagtacccgcactgcgaggagaagatggttatcatcaccaccaagagcgtgtccaggtaccgaggtcaggagcactgcctgcaccccaagctgcagagcaccaaacgcttcatcaagtggtacaacgcctggaacgagaagcgcagggtctacgaagaatagggtgaaaaatctctgatgggaaaactccagaccagttgggagacttgtgcaaaggactttgcagatttaaaaaaaaaaaaaaaaagcctttctttctcccaggcataagacacaagttatatattgttatgaagcactttttaccaacggtcagtttttacattttatagctgcgtgcgaaaggcttccagatgtgagacccatctctcttgtgctccagacttcatcacaggctgctttttatcgaaaaggggaaaactcatgcctttcctttttaagaaatgcttttttgtatttgtccatatgtcactatacatctgagctttataagcgcccgggaggaacaatgagcttggtggacacatttcattgcagtgttactccattcctagcttgggaagcttccgctcagaggtcctggcgcctcggcacagctgccacgggctctcctgggcttatggccggtcacagcctcagtgtgactctgcagtggcccctgtagccgggcaagcaggagcaggtctctctgcatctgttctttgaggaactcaagtttggttgccagaaaaatgtgcttcattcccccctggttaatttttacacaccctaggaaacatttccaagatcctgtgatggcgagacaaatgatccttaaagaagatgtggggtctttcccaacctgagactttctgaaaggttcacaggttcaatatttaatgcttcagaagcatgtgaggttcccaacactgtcagcaaaaaccttaggagaaaacttaaaaatatatgaatacatgcacaatacacagctacagactcacattctgttgacaagggaaaaccttcaaagcatgtttctctccctcaccacaacggaacatgcagtactaaagcaatatatttgtgattccccatgtaattcttcaatgttaaacagtgcagtcctctttcaaaagctaagatgaccatgcgccctttcctctgtacatatacccttaagaacgccccctccacacactgccctccagtatacgccgcattgtactgctgtgttatatgctacgtacatgtcagaaaccattagcattgcatgcaggtttcatattctttctaagatggaaagtaataaaatatatttgaaatgtaaaaaaaaaaaaaaaaaaaaaaaaaaaaa

SEQ ID NO: 11 - DNA, 20 bp, Artificial Sequence
  Description of Artificial Sequenceprimer
ccaagcgcttcatcaagtgg

SEQ ID NO: 12 - DNA, 21 bp, Artificial Sequence
  Description of Artificial Sequenceprimer
gcagcctgtgatgaagtctgg

SEQ ID NO: 13 - DNA, 24 bp, Artificial Sequence
  Description of Artificial Sequenceprimer
cagcctattcttcgtagaccctgc

SEQ ID NO: 14 - DNA, 478 bp, Artificial Sequence
  Description of Artificial Sequenceprobe
gtccaagtgtaagtgttcccggaaggggcccaagatccgctacagcgacgtgaagaagctggaaatgaagccaaagtacccacactgcgaggagaagatggttatcgtcaccaccaagagcatgtccaggtaccggggccaggagcactgcctgcaccctaagctgcagagcaccaaacgcttcatcaagtggtacaatgcctggaacgagaagcgcagggtctacgaagaatagggtggacgatcatggaaagaaaaactccaggccagttgagagacttcagcagaggactttgcagattaaaataaaagccctttctttctcacaagcataagacaaattatatattgctatgaagctcttcttaccagggtcagtttttacattttatagctgtgtgtgaaaggcttccagatgtgagatccagctcgcctgcgcaccagacttcattacaagtggctttttgctgggcggttg

SEQ ID NO: 15 - DNA, 2050 bp, Homo sapiens
  CDS (91)..(1176)
cgaggcggggagccgaggccgccgccagcgcgcagggagccccgagcccgagccccgccgcgaggccgttcgccggctgcccgcagcaccatggatctgcacctcttcgactacgccgagccaggcaacttctccgacataagctggccgtgcaacagcagcgactgcatcgtcgtggacaccgtgctgtgccccaacatgcccaacaaaagcgtgctgctgtacacgctgtccttcatttacatcttcatcttcgtgatcggcatgatcgccaactccgtggtggtctgggtgaacatccaggccaagaccaccggctacgacactcactgctacatcctcaacctggccatcgccgacctgtgggtggtcgtcaccatccccgtctgggtggtcagcctcgtgcagcataaccagtggcccatgggggagctcacgtgcaagatcacgcacctcatcttctccatcaacctgttcggcagcatcttcttcctcacgtgcatgagcgtggaccgctacctctccatcacctacttcgccagcacgtcgagccgcaggaagaaggtggttcgccgcgccgtctgtgtcctggtgtggctgctggccttctgcgtgtccctgcccgacacctactacctgaagaccgtcacgtcggcgtccaacaacgagacctactgccgctccttctaccccgagcacagcgtcaaggagtggctcatcagcatggagctggtctcggtggtcctgggcttcgccatccccttctgcgtcatcgccgtcttctactgcctgctggcccgcgccatctccgcgtccagcgaccaggagaagcagagcagccgaaagatcatcttctcctacgtggtggtcttcctcgtgtgctggctcccctaccacgtggtggtgctcctggacatcttctccatccttcactacatccccttcacctgccagctggagaacttcctcttcacggctctgcacgtcacgcagtgcctgtctctggtgcactgctgcgtcaaccccgtgctctatagcttcatcaaccgtaactacagatacgagctgatgaaggccttcatctttaagtactcggccaagacgggtctcaccaagctcatcgatgcctccagggtgtcggagacggagtactccgccttggagcaaaacgccaagtgacaggccccgcggggcctcggggacaagcgtgcgcgcttcctacacggcgctgggttgcatcgtctccaagagttcaagcaaggcgccttggggcttgaggcctgagtcgcgtgaagtggggaagcaggtgccaccgtgggcccttctcctctttgtccggctgccggggtggctgtcacggggccgtcgtgcctgcaggcgtgggccgtgctgcgtgaccctactacttactcgtccaagccggttgccggcgggctcgcctgcacttctgtaaaataggactttctgtgttccctggaggttttacttggtgacttgtatttaagttttaagactttattttctcattatcggcgtaccttataaatgtacttgaaagtttaaatatattttataaaaatcgtatgggcgctgtcacatacgatacatatacatacgctgacatatattcagagcgttgtagttttaactagtttgatttcagttttgactaaggatgacattaattgttagctgttttgaaattttatatatatatataaatataaatataaatatataaatatataaatatatgccggtctgggctgaaatgttttatttacgatagttttatatctgtgtggtgctttgctacctgcatgggatatgggacaagaaggacgctgtaaagcagtttgtgatattaattgtatcgtaaagttacattaaaaatgcgaaacgaagcggaacctctccaggctgcagcgccgcgcacaaagttttgaacagtcttatttcggagagttctctcaatttgtaagttatttttctttttttttttttttaataaagatttgtttttcctaaaaaaggcaaaaaaaaaaaaaaa

SEQ ID NO: 16 - protein, 362 aa, Homo sapiens
MDLHLFDYAEPGNFSDISWPCNSSDCIVVDTVLCPNMPNKSVLLYTLSFIYIFIFVIGMIANSVVVWVNIQAKTTGYDTHCYILNLAIADLWVVVTIPVWVVSLVQHNQWPMGELTCKITHLIFSINLFGSIFFLTCMSVDRYLSITYFASTSSRRKKVVRRAVCVLVWLLAFCVSLPDTYYLKTVTSASNNETYCRSFYPEHSVKEWLISMELVSVVLGFAIPFCVIAVFYCLLARAISASSDQEKQSSRKIIFSYVVVFLVCWLPYHVVVLLDIFSILHYIPFTCQLENFLFTALHVTQCLSLVHCCVNPVLYSFINRNYRYELMKAFIFKYSAKTGLTKLIDASRVSETEYSALEQNAK

SEQ ID NO: 17 - DNA, 1860 bp, Mus musculus
  CDS (89)..(1174)
cactgaaggagcctgcagcgctcaccgtcaggaaggcaaaccacagcccaggaagccctgaggtcacttggtcgctctcctcaagaccatggatgtgcacttgtttgactatgcagagcctggcaactactctgacatcaactggccatgtaacagcagcgactgcattgtggtggacactgtgcagtgtcccaccatgcctaacaagaacgtgcttctgtataccctctccttcatctacattttcatcttcgtgatcggcatgattgccaactctgtggtggtctgggtgaatatccaggctaagaccacaggctacgacacgcactgctacatcttgaacctggccattgcagacctgtgggtcgtcatcaccacccccgtctgggtggtcagtctcgtgcagcataaccagtggcccatgggggagctcacatgcaagatcacacacctcattttctccatcaacctctttgggagcatcttcttcctcgcctgcatgagcgtggaccgctatctctccatcacctacttcaccggcacctccagctataagaagaagatggtacgccgtgttgtatgcatcttggtgtggctgctggccttctttgtgtccctgcctgatacctactacctgaaggcggtcacatctgcttccaacaatgagacctactgcaggtccttctaccccgagcacagcatcaaggagtggctgatcggcatggagctggtctctgtcatcttgggctttgctgtccccttcactatcattgcgatcttctacttcctgctcgctagagccatgtcagcatcaggcgaccaggagaagcacagtagccggaagatcatcttctcctacgtggtggtcttcctggtatgttggctgccgtaccattttgtggttctgctggacatcttctccatcttacactacatcccgtttacctgtcagctggagaatgtgctctttacagcgttgcatgtcacccagtgcctgtccttggtgcactgctgtgtcaaccccgtgctctatagcttcatcaaccgcaactacaggtacgagctgatgaaggccttcatcttcaagtactcggccaaaacaggtctcaccaagctcattgatgcctccagagtgtcagagacagagtactctgccctggaacagaacaccaagtgatccatcattctgcagaggtcggggggacacgtgcatgttgcaaatggggcggctgggtcctgtggtttcttcaagaaagcaatgtagctttgggtctggttgcttgagtggtatgaagaggagaaagcatgtgaccatgcttcctgtctctcattctccagccaagatggctgccgcctggatgcccatcctgacagctggcagctggcagcaggcagggctgtgctgtgctgtgctgtgctgcgctgtgctgtgctgtgcagccagagctgcgtatcaaagccagcaccaggacaggctcttatggacatgtgtacagtagaatcttctgtgtttcttcaagtttttacttggtgactttttgtatttaagttttaagactttattttctcactatggatgtaccttataaatgtatttgaaagctaaatatattttaaatattgtatgggaggtgtaaggctgttgtattcagaccatgtaggcctcagattagctggacttgagttttgactaaggatgacattaattgttagctgatttgaaattatatatataaatataaaaatatatataaatttatgccagtctcggctgaaaggttttatttacaatagttttatatctgtgcaatgtttaatgctggcacagcatatgaaacaacaactgcccagcaatgcagt

SEQ ID NO: 18 - protein, 362 aa, Mus musculus
MDVHLFDYAEPGNYSDINWPCNSSDCIVVDTVQCPTMPNKNVLLYTLSFIYIFIFVIGMIANSVVVWVNIQAKTTGYDTHCYILNLAIADLWVVITTPVWVVSLVQHNQWPMGELTCKITHLIFSINLFGSIFFLACMSVDRYLSITYFTGTSSYKKKMVRRVVCILVWLLAFFVSLPDTYYLKAVTSASNNETYCRSFYPEHSIKEWLIGMELVSVILGFAVPFTIIAIFYFLLARAMSASGDQEKHSSRKIIFSYVVVFLVCWLPYHFVVLLDIFSILHYIPFTCQLENVLFTALHVTQCLSLVHCCVNPVLYSFINRNYRYELMKAFIFKYSAKTGLTKLIDASRVSETEYSALEQNTK

SEQ ID NO: 19 - DNA, 1895 bp, Canis familiaris
  CDS (1)...(1086)
  unsure 25, 27, 391, 796, 1065, 1077, 1079, 1081: The n at position 25, 27, 391, 796, 1065, 1077, 1079, and 1081 can be A, C, G, or T.
atggatctgcacctcttcgactacncngagccaggnaacttctcggacatcagctggccatgcaacagcagcgactgcatcgtggtggacacggtgatgtgtcccaacatgcccaacaaaagcgtcctgctctacacgctctccttcatttacattttcatcttcgtcatcggcatgattgccaactccgtggtggtctgggtgaatatccaggccaagaccacaggctatgacacgcactgctacatcttgaacctggccattgccgacctgtgggttgtcctcaccatcccagtctgggtggtcagtctcgtgcagcacaaccagtggcccatgggcgagctcacgtgcaaagtcacacacctcatcttctccatcaacctcttcagcngcattttcttcctcacgtgcatgagcgtggaccgctacctctccatcacctacttcaccaacacccccagcagcaggaagaagatggtacgccgtgtcgtctgcatcctggtgtggctgctggccttctgcgtgtctctgcctgacacctactacctgaagaccgtcacgtctgcgtccaacaatgagacctactgccggtccttctaccccgagcacagcatcaaggagtggctgatcggcatggagctggtctccgttgtcttgggctttgccgttcccttctccattatcgctgtcttctacttcctgctggccagagccatctcggcgtccagtgaccaggagaagcacagcagccggaagatcatcttctcctacgtggtggtcttccttgtctgctggntgccctaccacgtggcggtgctgctggacatcttctccatcctgcactacatccctttcacctgccggctggagcacgccctcttcacggccctgcatgtcacacagtgcctgtcgctggtgcactgctgcgtcaaccctgtcctctacagcttcatcaatcgcaactacaggtacgagctgatgaaggccttcatcttcaagtactcggccaaaacagggctcaccaagctcatcgatgcctccagagtntcngagacggagtactcngccttggagcanancnccaaatgatctgccctggagaggctctgggacgggtttacttgtttttgaacagggtgatgggccctatggttttctagagcaaagcaaagtagcttcgggtcttgatgcttgagtagagtgaagaggggagcacgtgccccctgcatccattntctctttctcttgatgacgcagctgtcatttggctgtgcgtgctgacagttttgcaacaggcagagctgtgtcgcacagcagtgctgtgcgtcagagccagctgaggacaggcttgcctggacttctgtaagataggattttctgtgtttcctgaattttttatatggtgatttgtatttaaattttaagactttattttctcactattggtgtaccttataaatgtatttgaaagttaaatatattttaaatattgtttgggaggcatagtgctgacatatattcagagtgttgtagttttaaggttagcgtgactttcagttttgactaaggatgacactaattgttagctgttttgaaattatatatatataaatatataaatatatgccagtcttggctgaaatgttttatttaccatagttttatatctgtgtggtgttttgtaccggcacgggatatggaacgaaaactgctttgtaatgcagtttgtgacattaatagtattgtaaagttacattttaaaataaacaaaaaactgttctggactgcaaatctgcacacacaacgaacagttgcatttcagagagttctctcaatttgtaagttatttttttttaataaagatttttgtttcctaaaaaaaaaaaaaaaaaaaa

SEQ ID NO: 20 - protein, 362 aa, Canis familiaris
  VARIANT (9)...(9): The amino acid at position 9 can be T, P, A, or S.
MDLHLFDYXEPGNFSDISWPCNSSDCIVVDTVMCPNMPNKSVLLYTLSFIYIFIFVIGMIANSVVVWVNIQAKTTGYDTHCYILNLAIADLWVVLTIPVWVVSLVQHNQWPMGELTCKVTHLIFSINLFSXIFFLTCMSVDRYLSITYFTNTPSSRKKMVRRVVCILVWLLAFCVSLPDTYYLKTVTSASNNETYCRSFYPEHSIKEWLIGMELVSVVLGFAVPFSIIAVFYFLLARAISASSDQEKHSSRKIIFSYVVVFLVCWXPYHVAVLLDIFSILHYIPFTCRLEHALFTALHVTQCLSLVHCCVNPVLYSFINRNYRYELMKAFIFKYSAKTGLTKLIDASRVSETEYSALEXXXK

SEQ ID NO: 21 - protein, 94 aa, Unknown
  Description of Unknown Organismmacaque
AALLLLLLALYTARVDGSKCKCSRKGPKIRYSDVKKLEMKPKYPHCEEKMVIITTKSVSRYRGQEHCLHPKLQSTKRFIKWYNAWNEKRRVYEE

SEQ ID NO: 22 - DNA, 285 bp, Unknown
  Description of Unknown Organismmacaque
gccgcgctactcctgctgctgctggcgctgtacaccgcgcgcgtggacgggtccaaatgcaagtgctcccggaagggacccaagattcgctacagcgacgtgaagaagctggaaatgaagccaaagtacccgcactgcgaggagaagatggttatcatcaccaccaagagcgtgtccaggtaccgaggtcaggagcactgcctgcaccccaagctgcagagcaccaaacgcttcatcaagtggtacaacgcctggaacgagaagcgcagggtctacgaagaatag